(19) United States
(12) Patent Application Publication
(10) Pub. No.: US 2012/0108855 A1
(43) Pub. Date: May 3, 2012

(54) INCREASED EXPRESSION OF TRANSHYDROGENASE GENES AND THEIR USE IN ETHANOL PRODUCTION

(75) Inventors: Lonnie O. Ingram, Gainesville, FL (US); Elliot N. Miller, Houston, TX (US); Laura R. Jarboe, Ames, IA (US)

(73) Assignee: UNIVERSITY OF FLORIDA RESEARCH FOUNDATION, INC., Gainesville, FL (US)

(21) Appl. No.: 13/320,633

(22) PCT Filed: Mar. 15, 2010
(86) PCT No.: PCT/US10/00769

§ 371 Date: Jan. 12, 2012

Related U.S. Application Data
(60) Provisional application No. 61/178,672, filed on May 15, 2009.
(60) Provisional application No. 61/235,340, filed on Aug. 19, 2009.
(60) Provisional application No. 61/292,094, filed on Jan. 4, 2010.

(51) Int. Cl. C07C 31/08; C12P 7/06; C12N 1/21

(52) U.S. Cl. 568/840; 435/252.3; 435/252.33; 435/252.31; 435/252.32; 435/161

(57) ABSTRACT

The invention provides isolated or recombinant ethanologenic bacteria that have increased expression of transhydrogenase genes and methods of preparation. The invention also provides methods of producing ethanol using the bacterium and corresponding kits.

### RELATED APPLICATIONS

This application claims priority to U.S. provisional application Ser. No. 61/178,672, filed May 15, 2009, U.S. provisional application Ser. No. 61/235,340, filed Aug. 19, 2009, and U.S. provisional application Ser. No. 61/292,094, filed Jan. 4, 2010, the entire disclosures of which are incorporated herein by this reference.

### GOVERNMENT SPONSORED RESEARCH

This invention was made with United States Government support under Contract Nos. US DOE FG02-96ER20222, DE-FG36-08GO88142, and DE-FC36-GO17058, awarded by the U.S. Department of Energy. The Government has certain rights in this invention.

### BACKGROUND OF THE INVENTION

A wide variety of fermentation products can be made using sugars from lignocellulosic biomass as a substrate (9, 13, 16, 37). Prior to fermentation, however, the carbohydrate polymers cellulose and hemicellulose must be converted to soluble sugars using a combination of chemical and enzymatic processes (38, 41). Chemical processes are accompanied by side reactions that produce a mixture of minor products such as alcohols, acids, and aldehydes that have a negative effect on the metabolism of microbial biocatalysts. Alcohols (catechol, syringol, etc.) have been shown to act by permeabilizing the cell membrane and toxicity correlated well with the hydrophobicity of the molecule (46). Organic acids (acetate, formate, etc.) are thought to cross the membrane in neutral form and ionize within the cytoplasm, inhibiting growth by collapsing the proton motive force (31, 45). The inhibitory mechanisms of aldehydes are more complex. Aldehydes can react to form products with many cellular constituents in addition to direct physical and metabolic effects (26, 34). In aggregate, these minor products from chemical pretreatments can retard cell growth and slow the fermentation of biomass-derived sugars (10, 30).

Furfural (a dehydration product of pentose sugars) is of particular importance. Furfural is a natural product of lignocellulosic decomposition. Furfural is also formed by the dehydration of pentose sugars during the depolymerization of cellulosic biomass under acidic conditions (21). This compound is an important contributor to toxicity of hemicellulose syrups, and increases the toxicity of other compounds (44). Furfural content in dilute acid hydrolysates of hemicellulose has been correlated with toxicity (22). Removal of furfural by lime addition (pH 10) rendered hydrolysates readily fermentable while re-addition of furfural restored toxicity (21). Furfural has also been shown to potentiate the toxicity of other compounds known to be present in acid hydrolysates of hemicellulose (44-46). Furfural has been reported to alter DNA structure and sequence (3, 17), inhibit glycolytic enzymes (6), and slow sugar metabolism (11).

The ability of fermenting organisms to function in the presence of these inhibitors has been researched extensively. Encapsulation of Saccharomyces cerevisiae in alginate has been shown to be protective and improve fermentation in acid hydrolysates of hemicellulose (36). Strains of S. cerevisiae have been previously described with improved tolerance to hydrolysate inhibitors (1, 19, 28). Escherichia coli (7), S. cerevisiae (2) and other microorganisms (4) have been shown to contain enzymes that catalyze the reduction of furfural to the less toxic product, furfuryl alcohol (46). In E. coli, furfural reductase activity appears to be NADPH-dependent (7). An NADPH-dependent furfural reductase was purified from E. coli although others may also be present. An NADPH-dependent enzyme capable of reducing 5-hydroxymethyl furfural (a dehydration product of hexose sugars) has been characterized in S. cerevisiae and identified as the ADH6 gene (33).

Accordingly, the ability to increase the tolerance to furfural by ethanol producing microorganisms would result in increased ethanol production by these microorganisms.

### SUMMARY OF THE INVENTION

The invention provides organisms for large-scale fuel production. Particularly, the invention provides bacteria that can grow and produce ethanol in the presence of increased furfural.

The invention provides for an isolated or recombinant ethanologenic bacterium having increased expression of at least one transhydrogenase gene as compared to a reference bacterium. In one embodiment, the transhydrogenase genes are pntA and pntB.

The invention also provides for an isolated or recombinant bacterium, wherein the bacterium has increased expression of pntA and pntB genes as compared to a reference bacterium.

In one embodiment, the bacterium has increased furfural tolerance as compared to the reference bacterium.

In another embodiment, the bacterium is a wild-type bacterium.

In another embodiment, the bacterium is ethanologenic.

In another embodiment, the bacterium exhibits increased ethanol production as compared to a reference bacterium.

In another embodiment, the bacterium exhibits increased ethanol production in the presence of furfural as compared to a reference bacterium.

In another embodiment, the bacterium has increased growth as compared to a reference bacterium.

In another embodiment, the bacterium has increased growth in the presence of furfural as compared to a reference bacterium.

In another embodiment, the bacterium has increased growth in the presence of furfural at concentrations between about 0.025% furfural to about 0.15% furfural.

In another embodiment, the bacterium has increased growth and increased ethanol production as compared to a reference bacterium.

In another embodiment, the bacterium has increased growth in the presence of a hydrolysate as compared to a reference bacterium.

In another embodiment, the bacterium has increased growth in the presence of a hydrolysate and the hydrolysate is derived from a product comprising a biomass, a hemicellulosic biomass, a lignocellulosic biomass or a cellulosic biomass.

In another embodiment, the expression of the pntA and pntB genes is increased or altered by modifying or adding a promoter that regulates the expression of the pntA and pntB genes.

In another embodiment, the expression of the pntA and pntB genes is increased or altered by placing the genes under the control of a different regulatory protein or under control of an additional regulatory protein.

In another embodiment, the bacterium is capable of producing ethanol as a primary fermentation product under anaerobic or microaerobic conditions.

In another embodiment, the bacterium is selected from the group consisting of Gram negative bacteria and Gram positive bacteria.

In another embodiment, the bacterium is selected from the group consisting of Gram negative bacteria and Gram positive bacteria, and the Gram-negative bacterium is selected from the group consisting of Escherichia, Zymomonas, Acinetobacter, Gluconobacter, Geobacter, Shewanella, Salmonella, Enterobacter and Klebsiella.

In another embodiment, the bacterium is selected from the group consisting of Gram negative bacteria and Gram positive bacteria, and the Gram-positive bacterium is selected from the group consisting of Bacillus, Clostridium, Corynebacterium, Lactobacillus, Lactococcus, Oenococcus, Streptococcus and Eubacterium.

In another embodiment, the bacterium is Escherichia coli.

In another embodiment, the bacterium is Klebsiella oxytoca.

The invention provides for an isolated or recombinant bacterium, wherein the activity of PntA and PntB proteins is increased as compared to a reference bacterium.

The invention provides for an isolated or recombinant bacterium, wherein the activity of PntA and PntB proteins is increased as compared to a reference bacterium and the bacterium has increased furfural tolerance as compared to the reference bacterium.

The invention also provides for an isolated or recombinant bacterium, wherein expression of the pntA and pntB genes is increased as compared to a reference bacterium, and wherein the bacterium has increased furfural tolerance as compared to the reference bacterium.

The invention also provides for an isolated or recombinant bacterium wherein the expression of the pntA and pntB genes or the activity of the PntA and PntB polypeptides is increased as compared to a reference bacterium, wherein furfural tolerance is increased as compared to the reference bacterium, wherein said bacterium is capable of producing ethanol, and wherein the bacterium is prepared by a process comprising the steps of:
(a) growing a candidate strain of the bacterium in the presence of furfural; and
(b) selecting bacterium that produces ethanol in the presence of furfural.

The invention also provides for a method for producing ethanol from a biomass, a hemicellulosic biomass, a lignocellulosic biomass, a cellulosic biomass or an oligosaccharide source comprising contacting the biomass, hemicellulosic biomass, lignocellulosic biomass, cellulosic biomass or oligosaccharide with any of the isolated or recombinant bacterium of the invention thereby producing ethanol from a biomass, hemicellulosic biomass, lignocellulosic biomass, cellulosic biomass or an oligosaccharide source.

Further, the invention provides for a method for producing ethanol from a biomass, a hemicellulosic biomass, a lignocellulosic biomass, a cellulosic biomass or an oligosaccharide source in the presence of furfural comprising contacting the biomass, hemicellulosic biomass, lignocellulosic biomass, cellulosic biomass or oligosaccharide with the isolated or recombinant bacterium of the invention, thereby producing ethanol from a biomass, hemicellulosic biomass, lignocellulosic biomass, cellulosic biomass or an oligosaccharide source.

In addition, the invention provides for ethanol produced by the methods of the invention.

The invention also provides for a kit comprising the isolated or recombinant bacterium of the invention.

### BRIEF DESCRIPTION OF THE DRAWINGS

FIGS. 1A-B Growth of various LY 180 strains harboring plasmids having different levels of expression of the sthA gene in the presence of furfural. Plots of cell density (optical density) versus furfural concentration are shown after a 24 hour period (FIG. 1A) and after a 48 hour period (FIG. 1B). Symbols: (▪), pTrc99a control without insert; (▴), pTrc99a-sthA forward direction uninduced sthA expression; (▴), pTrc99a-sthA forward direction sthA expression induced with 0.01 mM IPTG; and (♦), pTrc99a-sthA forward direction sthA expression induced with 0.1 mM IPTG.

FIGS. 2 A-B Growth of various LY 180 strains harboring plasmids having different levels of expression of the pntA and pntB genes in the presence of furfural. Plots of cell density (optical density) versus furfural concentration are shown after a 24 hour period (FIG. 2A) and after a 48 hour period (FIG. 2B). Symbols: (▪), pTrc99a control without insert; (▴), pTrc99a-pntAB forward direction uninduced pntAB expression; (▴), pTrc99a-pntAB forward direction pntAB expression induced with 0.01 mM IPTG; and (♦), pTrc99a-pntAB forward direction pntAB expression induced with 0.1 mM IPTG.

FIGS. 3 A-B present the effect of increased expressions of transhydrogenases (SthA and PntAB) on furfural tolerance. Cultures were grown for 48 hrs in AM1 minimal media containing 50 liter⁻¹ xylose and 1.0 g liter⁻¹ furfural. The empty vector served as a control. Inducer was added prior to inoculation. Cell density (in terms of optical density at 550 nm) is indicated for each of the strains at a 0.05% furfural concentration after a 24 hour period (FIG. 3A) and at a 0.10% furfural concentration after a 48 hour period (FIG. 3B). The control strains are shown as open bars. The strains harboring plasmids having different levels of expression of pntA are shown by hatched bars. The strains harboring different levels of expression of pntAB genes are shown as shaded bars.

FIGS. 4 A-B presents the pntA nucleic acid (FIG. 4A) and amino acid (FIG. 4B) sequences.

FIGS. 5 A-B presents the pntB nucleic acid (FIG. 5A) and amino acid (FIG. 5B) sequences.

FIGS. 6A-B present transcriptional and regulatory changes in LY180 following challenge with 0.5 g liter⁻¹ furfural challenge. Regulatory genes that were significantly perturbed were identified by NCA with a P-value cutoff of 0.05 relative to a null distribution. Regulators with increased activity are shown with a solid border, regulators with decreased activity are shown with a dashed border. Regulators that showed a mixed activity are shown in light grey (DcuR). FIG. 6A presents a partial regulator-gene response map. Representative genes that were perturbed greater than 2-fold are shown, with solid (dotted) indicating genes with increased (decreased) expression. Solid lines indicate activation by the connected regulator, dashed lines indicate repression. The direction of perturbation for DcuR is unclear and this regulator is shown in light gray. FIG. 6B presents a model illustrating the mechanism of growth inhibition by furfural. The addition of furfural induces two NADPH-dependent oxido-reductases (YqhD and DkgA) that compete with essential biosynthetic reactions for NADPH. Assimilation of sulfate requires 4 NADPH per cysteine. Secondary consequences include depletion of sulfur amino acids and a cascade of events from stalled translation and accumulation of many non-sulfur building block intermediates to a more general stress response, as evidenced by perturbation of the stringent factor SF and biosynthesis regulators ArgR, PurR and RutR.

FIG. 7 shows addition of furfural increased expression of genes other than hisG in the histidine pathway. Ratios for changes are shown in parentheses. Abbreviation: ACR, aminoimidazole carboxamide ribonucleotide.

FIG. 8 shows furfural increased expression of genes concerned with sulfur assimilation into cysteine and methionine. Pathways for the synthesis of threonine and isoleucine from aspartate are included for comparison. Genes up-regulated by 1.5-fold or greater are shown with a positive sign. Genes down-regulated by 1.5-fold or greater are shown with a negative sign.

All others are shown in black with no assigned positive or negative sign.

FIG. 9 presents the effect of media supplements on growth in the presence of 1 g liter⁻¹ furfural. Cultures are compared after incubation for 48 hours (AM1 medium, 50 g liter⁻¹ xylose, 37° C.). FIG. 9A presents addition of individual amino acids (0.1 mM each). FIG. 9B presents addition of amino acids (0.5 mM each). FIG. 9C presents addition of cysteine. FIG. 9D presents addition of alternative sulfur sources.

FIG. 10 is Table 1 and presents bacterial strains, plasmids, and primers.

FIG. 11 is Table 2 and presents genes perturbed greater than 2-fold in response of LY180 to treatment with 0.5 g liter⁻¹ furfural, sorted by functional group.

FIG. 12 is Table 3 and presents genes with changes in expression ratios of five-fold or greater in response to added furfural (0.5 g liter⁻¹). Some of these genes also changed more than two-fold in response to the addition of water in a control experiment (marked with an asterisk).

FIG. 13 is Table 4 and presents regulators significantly (P<0.05) perturbed in the LY180 furfural response relative to a null distribution, as determined by NCA.

FIG. 14. Effect of 5-HMF on anaerobic growth and fermentation. Cells were grown in AM1 mineral salts media with xylose (100 g l⁻¹ xylose). A. Cell mass during growth with 1.0 g l⁻¹ 5-HMF; B. Ethanol production during fermentation with 1.0 g l⁻¹ 5-HMF; C. Reduction of 5-HMF (1.0 g l⁻¹) during fermentation; D. Cell mass during growth with 2.5 g l⁻¹ 5-HMF; E. Ethanol production during fermentation with 2.5 g l⁻¹ 5-HMF; F. Reduction of 5-HMF (2.5 g l⁻¹ 5-HMF) during fermentation. Parallel fermentations without 5-HMF are included (dashed lines) in panels A and B for comparison. All data are plotted as a mean with standard deviation (n=3). Symbols for all: □, LY180; and , EMFR9.

FIG. 15. Effect of YqhD and DkgA on the in vitro reduction of 5-HMF and on 5-HMF tolerance. A. Specific activity for 5-HMF reduction in vitro. Activity was measured in lysed cell extracts (2 mM NADPH, 20 mM 5-HMF). B. Effect of yqhD and dkgA expression from plasmids on the cell yield of EMFR9 (resistant mutant). Experiments were performed in tube cultures with AM1 medium containing 50 g l⁻¹ xylose and 1.0 g 1⁻¹ 5-HMF (48 h incubation). Note that inclusion of kanamycin for plasmid maintenance lowers 5-HMF tolerance. Induced (Ind.) were grown with 0.1 mM IPTG. C. Effect of yqhD and dkgA deletions on the cell yield of LY180 (parent). Experiments were performed in tube cultures with AM1 medium containing 50 g l⁻¹ xylose and 2.5 g 1⁻¹ 5-HMF (48 h incubation). All data are plotted as a mean with standard deviation (n=4).

FIG. 16. Effect of pntAB expression from plasmids on 5-HMF tolerance. Experiments were conducted using the Bioscreen C growth curve analyzer with AM1 medium containing 50 g l⁻¹ xylose and 5-HMF as indicated. All data are plotted as a mean with standard deviation (n=10). Connecting points have been omitted for clarity. A. No supplement; B. Supplemented with 0.9 g l⁻¹ 5-HMF; and C. Supplemented with 1.8 g l⁻¹ 5-HMF. Symbols for all: Δ, LY180 (pTrc99a-control); ∘, LY180 (pTrc99a-pntAB) uninduced; , LY180 (pTrc99a-pntAB) induced with 0.01 mM IPTG.

FIG. 17. Effect of L-cysteine on 5-HMF tolerance of LY180. Experiments were performed in tube cultures with AM1 medium containing 50 g l⁻¹ xylose and 5-HMF (24 h incubation). Cultures were supplemented with filter-sterilized L-cysteine as indicated. All data are plotted as a mean with standard deviation (n=4). A. 1.0 g l⁻¹ 5-HMF; B. 2.0 g 1⁻¹ 5-HMF.

### DETAILED DESCRIPTION OF THE INVENTION

### I. Definitions

As used herein, “isolated” means free from contamination by other bacteria. An isolated bacterium can exist in the presence of a small fraction of other bacteria which do not interfere with the properties and function of the isolated bacterium. An isolated bacterium will generally be at least 30%, 40%, 50%, 60%, 70%, 80%, 85%, 90%, 95%, 98%, or 99% pure. Preferably, an isolated bacterium according to the invention will be at least 98% or at least 99% pure.

As used herein, “bacterium” includes “non-recombinant bacterium”, “recombinant bacterium” and “mutant bacterium”.

As used herein, “non-recombinant bacterium” includes a bacterial cell that does not contain heterologous polynucleotide sequences, and is suitable for further modification using the compositions and methods of the invention, e.g. suitable for genetic manipulation, e.g., which can incorporate heterologous polynucleotide sequences, e.g., which can be transfected. The term is intended to include progeny of the cell originally transfected. In particular embodiments, the cell is a Gram-negative bacterial cell or a Gram-positive bacterial cell.

As used herein, “recombinant” as it refers to bacterium, means a bacterial cell that is suitable for, or subjected to, genetic manipulation, or incorporates a heterologous polynucleotide sequence, or that has been treated such that a native polynucleotide sequence has been mutated or deleted.

As used herein, “mutant” as it refers to bacterium, means a bacterial cell that is not identical to a reference bacterium, as defined herein below.

A “mutant” bacterium includes a “recombinant” bacterium.

As used herein, “ethanologenic” means the ability of a bacterium to produce ethanol from a carbohydrate as a primary fermentation product. The term is intended to include naturally occurring ethanologenic organisms and ethanologenic organisms with naturally occurring or induced mutations.

The term “non-ethanologenic” means the inability of a bacterium to produce ethanol from a carbohydrate as a primary fermentation product. The term is intended to include microorganisms that produce ethanol as the minor fermentation product comprising less than 40% of total non-gaseous fermentation products.

As used herein, “ethanol production” means the production of ethanol from a carbohydrate as a primary fermentation product.

As used herein, “capable of producing ethanol” means capable of “ethanol production” as defined herein.

The terms “fermenting” and “fermentation” mean the degradation or depolymerization of a complex sugar and bioconversion of that sugar residue into ethanol, acetate and succinate. The terms are intended to include the enzymatic process (e.g. cellular or acellular, e.g. a lysate or purified polypeptide mixture) by which ethanol is produced from a carbohydrate, in particular, as a primary product of fermentation.

The terms “primary fermentation product” and “major fermentation product” are used herein interchangeably and are intended to include non-gaseous products of fermentation that comprise greater than about 50% of total non-gaseous product. The primary fermentation product is the most abundant non-gaseous product. In certain embodiments of the invention, the primary fermentation product is ethanol.

The term “minor fermentation product” as used herein is intended to include non-gaseous products of fermentation that comprise less than 40% of total non-gaseous product. In certain embodiments of the invention, the minor fermentation product is ethanol.

The term “simultaneous saccharification and fermentation” or “SSF” is intended to include the use of one or more recombinant hosts (or extracts thereof, including purified or unpurified extracts) for the contemporaneous degradation or depolymerization of a complex sugar and bioconversion of that sugar residue into ethanol by fermentation. SSF is a well-known process that can be used for breakdown of biomass to polysaccharides that are ultimately convertible to ethanol by bacteria. Reflecting the breakdown of biomass as it occurs in nature, SSF combines the activities of fungi (or enzymes such as cellulases extracted from fungi) with the activities of ethanologenic bacteria (or enzymes derived therefrom) to break down sugar sources such as lignocellulose to simple sugars capable of ultimate conversion to ethanol. SSF reactions are typically carried out at acid pH to optimize the use of the expensive fungal enzymes.

The term “sugar” is intended to include any carbohydrate source comprising a sugar molecule(s). Such sugars are potential sources of sugars for depolymerization (if required) and subsequent bioconversion to acetaldehyde and subsequently to ethanol by fermentation according to the products and methods of the present invention. Sources of sugar include starch, the chief form of fuel storage in most plants, hemicellulose, and cellulose, the main extracellular structural component of the rigid cell walls and the fibrous and woody tissues of plants. The term is intended to include monosaccharides, also called simple sugars, oligosaccharides and polysaccharides. In certain embodiments, sugars include, e.g., glucose, xylose, arabinose, mannose, galactose, sucrose, and lactose. In other embodiments, the sugar is glucose.

As used herein, “PntAB”, means a pyridine nucleotide transhydrogenase. pntAB, also known as pntA and pntB, refer to the genes corresponding to the PntAB transhydrogenase whereas the term PntAB refers to a pntAB gene product.

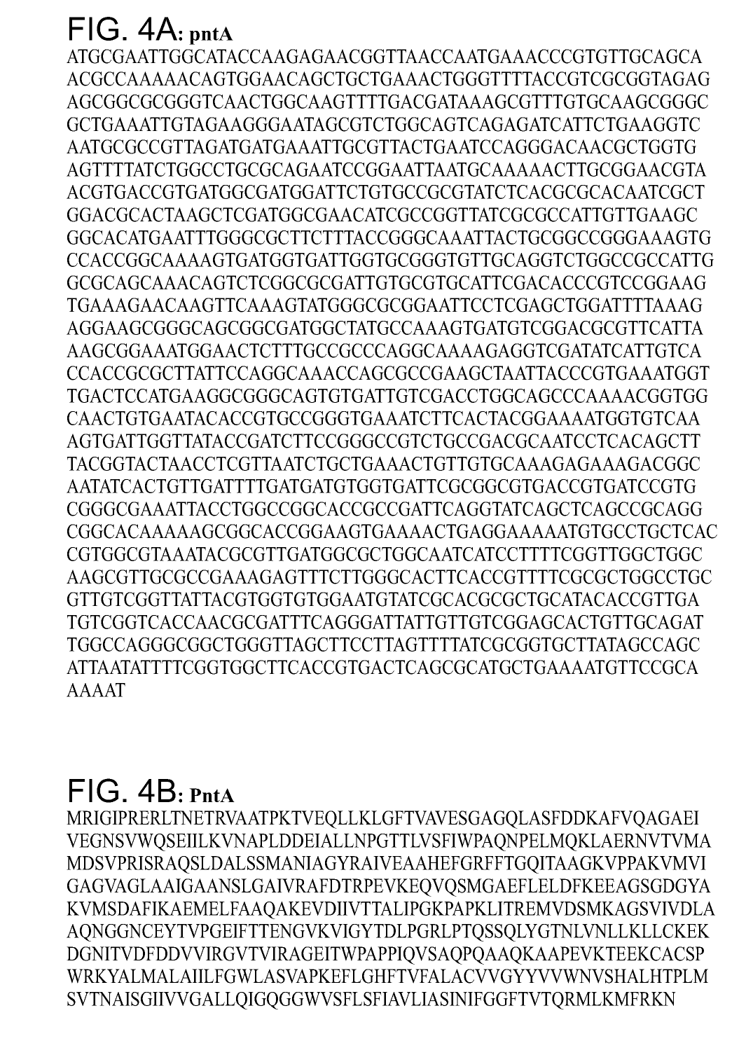

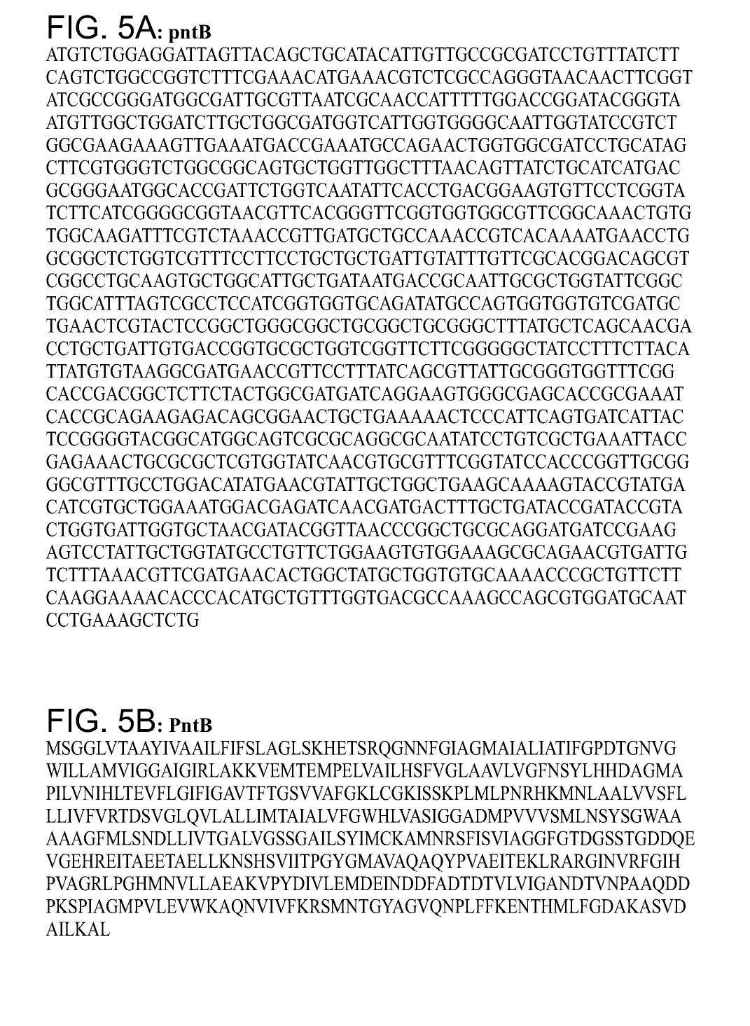

The amino and nucleic acid sequences corresponding to the pntA and pntB genes are presented in FIGS. 4 A-B and 5 A-B, respectively.

As used herein, “SthA”, means a cytoplasmic transhydrogenase. sthA refers to the gene corresponding to the SthA transhydrogenase whereas the term SthA refers to the sthA gene product.

As used herein, “mutant nucleic acid molecule” or “mutant gene” is intended to include a nucleic acid molecule or gene having a nucleotide sequence which includes at least one alteration (e.g., substitution, insertion, deletion) such that the polypeptide or polypeptide that can be encoded by the mutant exhibits an activity or property that differs from the polypeptide or polypeptide encoded by the wild-type nucleic acid molecule or gene.

As used herein, “mutation” as it refers to a nucleic acid molecule or gene means alteration, insertion or deletion of a nucleic acid or a gene, or an increase or decrease in the level of expression of a nucleic acid or a gene, wherein the increase or decrease in expression results in a respective increase or decrease in the expression of the polypeptide that can be encoded by the nucleic acid molecule or gene. A mutation also means a nucleic acid molecule or gene having a nucleotide sequence which includes at least one alteration (e.g., substitution, insertion, deletion) such that the polypeptide or polypeptide that can be encoded by the mutant exhibits an activity or property that differs from the polypeptide or polypeptide encoded by the wild-type nucleic acid molecule or gene.

As used herein, “mutant protein” or “mutant protein or amino acid sequence” is intended to include an amino acid sequence which includes at least one alteration (e.g., substitution, insertion, deletion) such that the polypeptide or polypeptide that can be encoded by the mutant amino acid sequence exhibits an activity or property that differs from the polypeptide or polypeptide encoded by the wild-type amino acid sequence.

As used herein, “mutation” as it refers to a protein or amino acid sequence means alteration, insertion or deletion of an amino acid of an amino acid sequence, or an increase or decrease in the level of expression of an amino acid sequence, wherein the increase or decrease in expression results in a increase or decrease in the expression of the polypeptide that can be encoded by amino acid sequence. A mutation also means a protein or amino acid sequence having an amino acid sequence which includes at least one alteration (e.g., substitution, insertion, deletion) such that the polypeptide or polypeptide that can be encoded by the mutant exhibits an activity or property that differs from the polypeptide or polypeptide encoded by the wild-type amino acid sequence.

As used herein, “fragment” or “subsequence” is intended to include a portion of parental or reference nucleic acid sequence or amino acid sequence, or a portion of polypeptide or gene, which encodes or retains a biological function or property of the parental or reference sequence, polypeptide or gene.

A “mutant” bacterium includes a bacterium comprising a “mutation” as defined hereinabove.

As used herein, “reference” or “reference bacterium” includes, at least, a wild-type bacterium and a parental bacterium.

As used herein, “wild-type” means the typical form of an organism or strain, for example a bacterium, gene, or characteristic as it occurs in nature, in the absence of mutations. “Wild type” refers to the most common phenotype in the natural population. Wild type is the standard of reference for the genotype and phenotype.

As used herein, “parental” or “parental bacterium” refers to the bacterium that gives rise to a bacterium of interest.

A “gene,” as used herein, is a nucleic acid that can direct synthesis of an enzyme or other polypeptide molecule, e.g., can comprise coding sequences, for example, a contiguous open reading frame (ORF) that encodes a polypeptide, a subsequence thereof, or can itself be functional in the organism. A gene in an organism can be clustered in an operon, as defined herein, wherein the operon is separated from other genes and/or operons by intergenic DNA. Individual genes contained within an operon can overlap without intergenic DNA between the individual genes. In addition, the term “gene” is intended to include a specific gene for a selected purpose. A gene can be endogenous to the host cell or can be recombinantly introduced into the host cell, e.g., as a plasmid maintained episomally or a plasmid (or fragment thereof) that is stably integrated into the genome. A heterologous gene is a gene that is introduced into a cell and is not native to the cell.

The term “nucleic acid” is intended to include nucleic acid molecules, e.g., polynucleotides which include an open reading frame encoding a polypeptide, a subsequence thereof, and can further include non-coding regulatory sequences, and introns. In addition, the terms are intended to include one or more genes that map to a functional locus. In addition, the terms are intended to include a specific gene for a selected purpose. In one embodiment, the term gene includes any gene encoding a transhydrogenase, including but not limited to pntA and pntB. In one embodiment, the gene or polynucleotide segment is involved in at least one step in the bioconversion of a carbohydrate to ethanol. A gene in an organism can be clustered in an operon, as defined herein, wherein the operon is separated from other genes and/or operons by intergenic DNA.

As used herein, “increasing” or “increases” or “increased” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, for example, as compared to the level of expression of the pntA and pntB genes, in a bacterium having an increased expression of the pntA and pntB genes, as compared to a reference bacterium.

As used herein, “increasing” or “increases” or “increased” also means increases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of expression of the pntA and pntB genes in a bacterium, having an increased expression of the pntA and pntB genes, as compared to a reference bacterium.

As used herein, “decreasing” or “decreases” or “decreased” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the decreased level of expression of the pntA and pntB genes in a bacterium, as compared to a reference bacterium.

As used herein, “decreasing” or “decreases” or “decreased” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of expression of the pntA and pntB genes in a bacterium, as compared to a reference bacterium.

“Decreased” or “reduced” also means eliminated such that there is no detectable level of activity, expression, etc., for example no detectable level of expression of the pntA and pntB genes or no detectable activity of the PntA and pntB proteins.

As used herein, “activity” refers to the activity of a gene, for example the level of transcription of a gene. “Activity” also refers to the activity of an mRNA, for example, the level of translation of an mRNA. “Activity” also refers to the activity of a protein, for example PntA and PntB.

An “increase in activity” includes an increase in the rate and/or the level of activity.

As used herein, “expression” as in “expression of pntA and pntB” refers to the expression of the protein product of the pntA and pntB genes. As used herein, “expression” as in “expression of pntA and pntB” also refers to the expression of detectable levels of the mRNA transcript corresponding to the pntA and/or pntB genes.

“Altering”, as it refers to expression levels, means decreasing expression of a gene, mRNA or protein of interest, for example the pntA and/or pntB genes.

As used herein, “not expressed” means there are no detectable levels of the product of a gene or mRNA of interest, for example, pntA and/or pntB genes.

As used herein “eliminate” means decrease to a level that is undetectable.

As used herein, “tolerance of furfural” means the ability of an ethanologenic bacterium to grow or produce ethanol in the presence of furfural, for example furfural at a concentration of 0.1 g liter⁻¹ or more (e.g. 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.5, 3.0 g liter⁻¹ or more). Tolerance of furfural also means the ability of an ethanologenic bacterium to grow or produce ethanol in the presence of furfural at a level that is increased as compared to the level of growth or ethanol production by a wild-type bacterium or a parental bacterium.

As used herein, “in the presence of” as it applies to the presence of furfural, means maintenance of a bacterium in the presence of at least 0.1 g liter⁻¹ or more (e.g. 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.5, 3.0 g liter⁻¹ or more) of furfural.

As used herein, “in the absence of” as it applies to the absence of furfural means maintenance of a bacterium in media that contains 0.1 g liter⁻¹ or less, including no detectable level, of furfural.

As used herein, “growth” means an increase, as defined herein, in the number or mass of a bacterium over time.

As used herein, “hemicellulose hydrolysate” includes but is not limited to hydrolysate derived from a biomass, a hemicellulosic biomass, a lignocellulosic biomass or a cellulosic biomass.

As used herein, “derived from” means originates from.

The term “Gram-negative bacterial cell” is intended to include the art-recognized definition of this term. Exemplary Gram-negative bacteria include Acinetobacter, Gluconobacter, Zymomonas, Escherichia, Geobacter, Shewanella, Salmonella, Enterobacter and Klebsiella.

The term “Gram-positive bacteria” is intended to include the art-recognized definition of this term. Exemplary Gram-positive bacteria include Bacillus, Clostridium, Corynebacterium, Lactobacillus, Lactococcus, Oenococcus, Streptococcus and Eubacterium.

The term “amino acid” is intended to include the 20 alpha-amino acids that regularly occur in proteins. Basic charged amino acids include arginine, asparagine, glutamine, histidine and lysine. Neutral charged amino acids include alanine, cysteine, glycine, isoleucine, leucine, methionine, phenylalanine, proline, serine, threonine, tryptophan, tyrosine, and valine. Acidic amino acids include aspartic acid and glutamic acid.

As used herein, “selecting” refers to the process of determining that an identified bacterium produces ethanol in the presence of furfural.

As used herein, “identifying” refers to the process of assessing a bacterium and determining that the bacterium produces ethanol in the presence of furfural.

As used herein, “increasing concentrations of furfural” means increments from 0 to 5 g/L, for example, 1 μg/L increments, 1 mg/L increments or 1 g/L increments.

In this disclosure, “comprises,” “comprising,” “containing” and “having” and the like have the open-ended meaning ascribed to them in U.S. patent law and mean “includes,” “including,” and the like.

As used herein, the term “transhydrogenase” refers to an enzyme which catalyzes the interconversion of reducing equivalents between nicotinamide adenine dinucleotide cofactors NAD(H) and NADP(H).

As used herein, the term “transhydrogenase gene” refers to a gene or genes whose product(s) is/are a transhydrogenase.

As used herein, the term “5-hydroxymethyl furfural” or “5-HMF” is intended to mean an organic compound derived from dehydration of sugars having the structure:

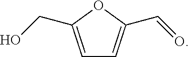

### II. Bacteria

The invention relates to bacteria suitable for degrading sugars for the formation of ethanol. The bacteria have improved ethanol production capabilities, particularly in medium containing furfural and/or 5-HMF. The capacity for improved ethanol production is related to the selected increased expression of transhydrogenase genes which increases the cells' tolerance of furfural and/or 5-HMF during sugar digestion and fermentation.

Accordingly, the invention provides ethanologenic bacteria that have increased expression of at least one transhydrogenase gene as compared to a reference bacterium. In one aspect of the invention, the bacteria are isolated bacteria. In another aspect of the invention, the bacteria are recombinant bacteria. In yet another aspect of the invention, the transhydrogenase gene includes the pntA and pntB genes.

The invention also provides isolated or recombinant bacteria that have increased expression of the pntA and pntB genes. In one aspect of the invention, the isolated bacteria of the invention include a wild-type bacterium.

The bacteria of the invention may be characterized by their increased growth. The bacteria of the invention are further characterized by their ability to grow in increased concentrations of furfural and/or 5-HMF. Accordingly, in other aspects of the invention, the isolated or recombinant bacteria have increased growth as compared to a reference bacterium, or increased furfural and/or 5-HMF tolerance as compared to reference bacterium, or increased growth in the presence of furfural and/or 5-HMF as compared to a reference bacterium, or increased growth in the presence of furfural and/or 5-HMF, for example, at furfural concentrations between about 0.025% furfural to about 0.15% furfural, or 5-HMF at concentrations between about 0.025% 5-HMF to about 0.15% 5-HMF.

The bacteria of the invention may also be characterized by their ability to produce ethanol as the primary fermentation product from a sugar source. Although furfural and 5-HMF typically inhibit the growth of ethanologenic bacteria during cellulosic digestion and fermentation to ethanol, the bacteria of the invention can produce ethanol in increased concentrations of furfural and/or 5-HMF. Accordingly, other aspects of the invention include bacteria that are ethanologenic, or exhibit increased ethanol production as compared to a reference bacterium, or exhibit increased ethanol production in the presence of furfural and/or 5-HMF as compared to a reference bacterium, or are capable of producing ethanol as a primary fermentation product under anaerobic or microaerobic conditions, or increased growth and increased ethanol production in the presence of furfural and/or 5-HMF as compared to a reference bacterium.

Further aspects of the invention include bacteria having increased growth in the presence of a hydrolysate as compared to a reference bacterium. The invention provides for a variety of hydrolysates including but not limited to hydrolysate derived from a biomass, a hemicellulosic biomass, a lignocellulosic biomass or a cellulosic biomass.

to a reference bacterium. For example, the promoter is altered by art-accepted methods including but not limited to replacement of the promoter by a different promoter or modification of the promoter by, for example, inserting, substituting, duplicating or removing nucleic acids or by inserting, substituting, duplicating or removing regulatory elements or motifs in the promoter. Accordingly, in one aspect of the invention, the expression of the transhydrogenase genes such as pntA and pntB genes of the invention are increased by methods known in the art including but not limited to modifying or adding a promoter that regulates the gene expression as compared to a reference bacterium.

The invention provides for methods of altering regulation of the pntA and pntB gene(s), by methods known in the art, including but not limited to placing the pntA and pntB gene(s) under the control of a different regulatory protein or under the control of an additional regulatory protein as compared to the reference bacterium. In one embodiment, the regulatory protein is a repressor. In an alternative embodiment, the regulatory protein is an inducer.

The bacteria of the invention may be non-recombinant or recombinant. The bacterium of the invention are selected from the group consisting of Gram-negative bacteria and Gram-positive bacteria, wherein the Gram-negative bacterium is selected from the group consisting of Acinetobacter, Gluconobacter, Zymomonas, Escherichia, Geobacter, Shewanella, Salmonella, Enterobacter and Klebsiella and the Gram-positive bacterium is selected from the group consisting of Bacillus, Clostridium, Corynebacterium, Lactobacillus, Lactococcus, Oenococcus, Streptococcus and Eubacterium. In one aspect, the bacterium of the invention is Escherichia coli and in another aspect, the bacterium of the invention is Klebsiella oxytoca.

It is understood to one of skill in the art that the protein activity associated with the increased transhydrogenase genes such as the pntA and pntB genes of the bacteria of the invention can be increased or altered. For example, the amino acids in the gene products can be substituted, added or deleted to a certain degree without substantially affecting the function of a gene product as compared with a naturally-occurring gene product. Accordingly, the invention also provides for isolated or recombinant bacteria, wherein the activity of the PntA and PntB proteins is increased or altered as compared to a reference bacterium. In one embodiment, such bacteria have increased furfural and/or 5-HMF tolerance as compared to a reference bacterium.

The invention provides for bacteria which have an increased or altered expression of transhydrogenase genes such as, for example, pntA and pntB genes, and which can continue to grow in increased concentrations of furfural, as discussed above. Thus, the invention further provides for isolated or recombinant bacteria wherein the expression of the pntA and pntB genes is increased or altered as compared to a reference bacterium, and wherein the bacteria has increased furfural and/or 5-HMF tolerance as compared to the reference bacterium. Expression is increased or altered by methods known in the art, including but not limited to modification of the pntA or pntB gene (e.g. by inserting, substituting or removing nucleic acids or amino acids in the sequences encoding the genes).

The invention also provides for an isolated or recombinant bacterium wherein the expression of the pntA and pntB genes or the activity of the PntA and PntB polypeptides is increased as compared to a reference bacterium, wherein furfural and/or 5-HMF tolerance is increased as compared to the reference bacterium, wherein the isolated or recombinant bacterium is capable of producing ethanol, and wherein the isolated or recombinant bacterium is prepared by a process comprising the steps of (a) growing a candidate strain of the bacterium in the presence of furfural and/or 5-HMF; and (b) selecting bacterium that produces ethanol in the presence of furfural and/or 5-HMF.

The invention further provides microorganisms suitable for fermenting sugars for the production of ethanol in the presence of furfural and/or 5-HMF. Accordingly, the invention provides a microorganism having increased expression of transhydrogenase genes which are endogenous to the microorganism or which are recombinantly introduced into the microorganism as, for example, a plasmid maintained episomally or a plasmid (or fragment thereof) that is stably integrated into the genome.

### III. Methods of Making

The invention further relates to methods for producing bacteria which are suitable for fermenting sugars for the production of ethanol in the presence of furfural and/or 5-HMF, e.g., in the presence of increased furfural and/or 5-HMF. Thus, the invention provides an isolated or recombinant bacterium wherein the expression of the pntA and pntB genes or the activity of the PntAB polypeptides is increased as compared to a reference bacterium, wherein furfural and/or 5-HMF tolerance is increased as compared to a reference bacterium, wherein the bacterium is capable of producing ethanol, and wherein the bacterium is prepared by a process comprising the steps of growing the candidate strain of the bacterium in the presence of furfural and/or 5-HMF and selecting bacterium that produce ethanol in the presence of furfural and/or 5-HMF.

Methods of making recombinant ethanologenic microorganisms are known in the art of molecular biology. Suitable materials and methods and recombinant microorganisms are described, for example, in U.S. Pat. Nos. 7,026,152, 6,849,434, 6,333,181, 5,821,093; 5,482,846; 5,424,202; 5,028,539; 5,000,000; 5,487,989, 5,554,520, and 5,162,516 and in WO2003/025117 hereby incorporated by reference, and may be employed in carrying out the present invention. The bacterium of the invention described herein can be made by methods routinely performed by one of skill in the art and are demonstrated in the examples as set forth in the application.

### IV. Methods for Producing Ethanol

The bacteria of the present invention are suitable for degrading sugars for the production of ethanol. Accordingly, the present invention provides methods for producing ethanol from source which comprises contacting the source with the isolated or recombinant bacterium of the invention described above, thereby producing ethanol from the source. In a particular embodiment of the invention, the source may be selected from a group consisting of a biomass, hemicellulosic biomass, lignocellulosic biomass, cellulosic biomass or an oligosaccharide source, or any combination thereof.

The bacteria of the invention may be used to produce ethanol from sugars in the presence of increased concentrations of furfural and/or 5-HMF. Accordingly, the invention provides a method for producing ethanol from a source which comprises contacting the source in the presence of furfural and/or 5-HMF with the isolated or recombinant bacterium of the invention described above, thereby producing ethanol from the source. In a particular embodiment of the invention, the source may be selected from a group consisting of a biomass, hemicellulosic biomass, lignocellulosic biomass, cellulosic biomass or an oligosaccharide source, or any combination thereof.

The microorganisms of the invention are characterized by an ethanol production under anaerobic conditions. Wild type E. coli produces ethanol and acetate at a ratio of 1:1 during anaerobic growth. During stationary phase of growth, wild type E. coli produces lactate as the main product, and the fraction of ethanol in the total fermentation products is about 20%. The products in all these fermentations comprise various acids, thus leading to the term, mixed acid fermentation.

Typically, fermentation conditions are selected that provide an optimal pH and temperature for promoting the best growth kinetics of the producer host cell stain and catalytic conditions for the enzymes produced by the culture (Doran et al., (1993) Biotechnol. Progress. 9:533-538). A variety of exemplary fermentations conditions are disclosed in U.S. Pat. Nos. 5,487,989 and 5,554,520. For a non-limiting example, conditions including temperatures ranging from about 25 to about 40° C. and a pH ranging from about 4.5 to 8.0 may be selected. See, for example, U.S. Pat. Nos. 5,424,202 and 5,916,787, which are specifically incorporated herein by this reference.

The invention provides for an isolated or recombinant bacterium with increased expression of transhydrogenase genes or for an isolated or recombinant bacterium with an increased expression of the pntA and pntB genes as compared to a reference bacterium. This bacterium can be used for producing ethanol, and particularly for producing ethanol from a source such as, for example, biomass, hemicellulosic biomass, lignocellulosic biomass, cellulosic biomass or an oligosaccharide. Accordingly, the invention provides a method for producing ethanol from a biomass, a hemicellulosic biomass, a lignocellulosic biomass, a cellulosic biomass or an oligosaccharide source comprising contacting the biomass, hemicellulosic biomass, lignocellulosic biomass, cellulosic biomass or oligosaccharide source with the bacterium of the invention, thereby producing ethanol from a biomass, a hemicellulosic biomass, a lignocellulosic biomass, a cellulosic biomass or an oligosaccharide source. Such production may occur in the presence or absence of furfural and/or 5-HMF.

In accordance with the methods of the invention, the bacterium described herein degrade or depolymerize a cellulosic biomass or oligosaccharide source with the bacterium of the invention, thereby producing ethanol from a biomass, a hemicellulosic biomass, a lignocellulosic biomass, a cellulosic biomass such as an oligosaccharide source into a monosaccharide. Subsequently, the bacterium by virtue of the increased expression of transhydrogenase genes or in particular, the increased expression of the pntA and pntB genes they carry, catabolize the simpler sugars into ethanol by fermentation. This process of concurrent complex saccharide depolymerization into smaller sugar residues followed by fermentation is referred to as simultaneous saccharification and fermentation (SSF).

Currently, the conversion of a complex saccharide such as lignocellulose is a very involved, multi-step process. For example, the lignocellulose must first be degraded or depolymerized using acid hydrolysis. This is followed by steps that separate liquids from solids and these products are subsequently washed and detoxified to result in cellulose that can be further depolymerized and finally, fermented by a suitable ethanologenic host cell. In contrast, the fermenting of corn is much simpler in that amylases can be used to break down the corn starch for immediate bioconversion by an ethanologenic host in essentially a one-step process.

It will be appreciated by the skilled artisan that the bacterium and methods of the invention afford the use of more efficient processes for fermenting lignocellulose. For example, the method of the invention is intended to encompass a method that avoids acid hydrolysis altogether. Moreover, the microorganisms of the invention advantageously can ferment sugars in the presence of the increased concentrations of the toxin furfural.

One advantage of the invention is the ability to use a saccharide source that has been, heretofore, underutilized. Consequently, a number of complex saccharide substrates may be used as a starting source for depolymerization and subsequent fermentation using the recombinant bacteria and methods of the invention. Ideally, a recyclable resource may be used in the SSF process. Mixed waste office paper is a preferred substrate (Brooks et al., (1995) Biotechnol. Progress. 11:619-625; Ingram et al., (1995) U.S. Pat. No. 5,424,202), and is much more readily digested than acid pretreated bagasse (Doran et al., (1994) Biotech. Bioeng. 44:240-247) or highly purified crystalline cellulose (Doran et al. (1993) Biotechnol. Progress. 9:533-538). Glucanases, both endoglucanases and exoglucanases, contain a cellulose binding domain, and these enzymes can be readily recycled for subsequent fermentations by harvesting the undigested cellulose residue using centrifugation (Brooks et al., (1995) Biotechnol. Progress. 11:619-625). Such approaches work well with purified cellulose, although the number of recycling steps may be limited with substrates with a higher lignin content. Other substrate sources that are within the scope of the invention include any type of processed or unprocessed plant material, e.g., lawn clippings, husks, cobs, stems, leaves, fibers, pulp, hemp, sawdust, newspapers, etc.

The invention also provides for a kit comprising an isolated or recombinant bacterium of the invention as described above. This kit optionally provides instructions for use, such as, for example, instructions for producing ethanol in accordance with the methods and processes described herein. Such instructions optionally may describe producing ethanol in increased concentrations of furfural. In one embodiment, the kit comprises a sugar source.

### EXEMPLIFICATION

The invention is further illustrated by the following examples, which should not be construed as limiting. Throughout the examples, the following materials and methods are used unless otherwise stated.

### Material and Methods

Strain LY 168 has been previously described for the fermentation of sugars in hemicellulose hydrolysates. Several modifications were made to improve substrate range (restoration of lactose utilization, integration of an endoglucanase, and integration of cellobiose utilization) resulting in LY 180 (NRRL B-50239). Relevant characteristics for these strains are provided in the following Table 1. The linear fragments used for integration shown in Table 1 are deposited in GenBank.

TABLE 1

| Strain | | Reference |
|---|---|---|
| STRAINS | Relevant characteristics | of source |
| LY168 | frdA::(Zm frg celY_{Ec}FRT) ΔldhA::FRT | Jarboe et |
| | ΔadhE::(Zm frg estZ_{Pp} FRT) ΔackA::FRT | al 2007; |
| | rrlE::(pdc adhA adhB FRT) lacY::FRT | Yomano et |
| | ΔmgsA::FRT, | al. 2008 |
| LY180 | ΔfrdBC::(Zm frg celY_{Ec}) ΔldhA::(Zm | |
| | frg casAB_{Ko}) adhE::(Zm frg estZ_{Pp} FRT) | |
| | Δack::FRT rrlE::(pdc adhA adhB FRT) | |
| | ΔmgsA::FRT | |

LY 180 strains were grown on LB glucose ampicillin plates overnight and each were used to inoculate a tube of AM1 xylose to 3-4 OD. This culture was immediately used to inoculate thirteen 100 mm capped tubes containing 4 mL AM1 5% xylose, ampicillin, and an indicated concentration of furfural to 0.05 initial OD. Cultures were grown in a water bath at 37 C and OD 500 nm readings were taken after 24 and 48 hours.

### Example 1

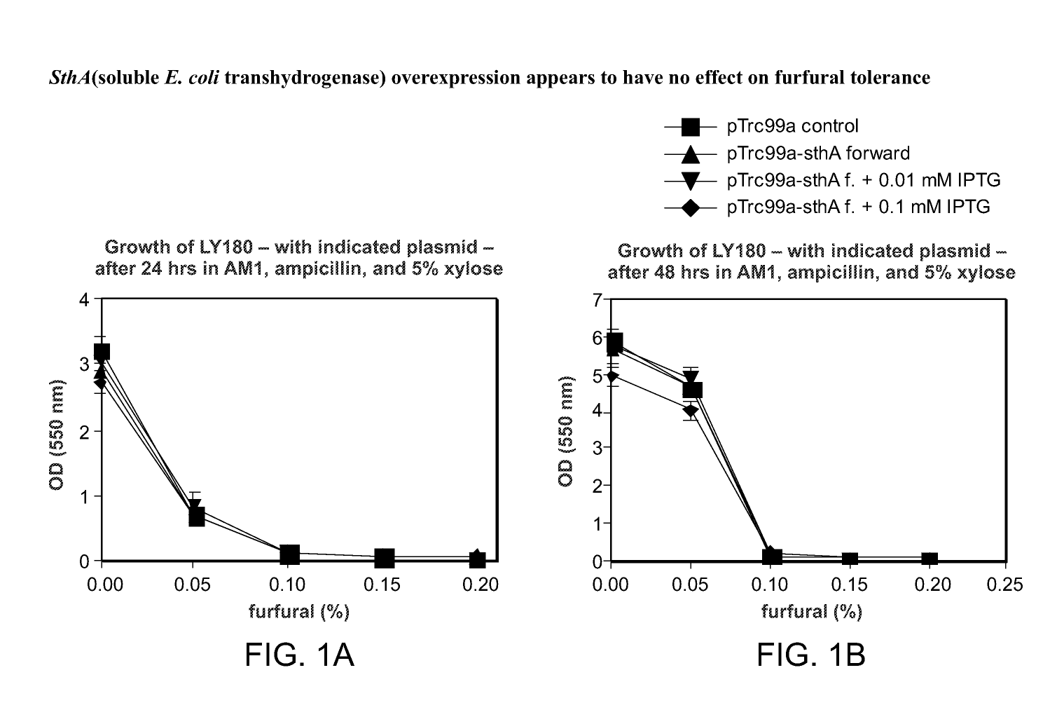

SthA is a cytoplasmic transhydrogenase with kinetic characteristics that promote function primarily in the direction of NADPH oxidation (52). The E. coli cytoplasmic transhydrogenase SthA gene was cloned into pTrc99a and confirmed by sequencing. LY180 strains were modified with these plasmids as indicated in FIGS. 1 A-B. Functionality of the cloned gene was confirmed by in vitro assays. The growth of the modified LY180 strains in AM1, ampicillin, and 5% xylose was compared after 24 hours and after 48 hours. Optical density was plotted versus increasing furfural concentrations. The empty vector served as a control. Inducer was added prior to inoculation.

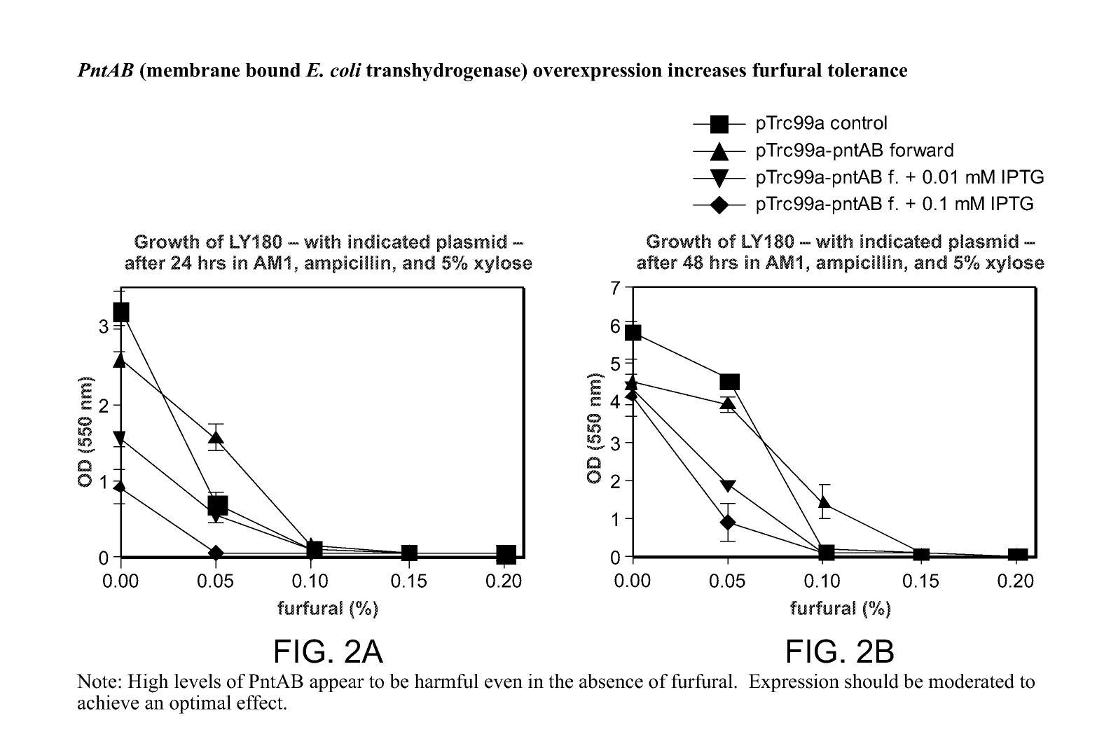

Upon induction with 0.1 mM IPTG, activity was found to increase from approximately 1.0 nmol min⁻¹ mg protein⁻¹ to 18 nmol min⁻¹ mg protein⁻¹. Expression of the sthA gene from a plasmid did not alter furfural tolerance with or without IPTG induction (FIG. 1). The results in FIG. 1A-1B demonstrate that after 24 hours (FIG. 1A) and after 48 hours (FIG. 2B), respectively, all the LY180 strains showed substantially similar decreases in growth with increasing furfural concentration regardless of the level of sthA gene expression. The over-expression of sthA gene appeared to have no effect on furfural tolerance.

### Example 2

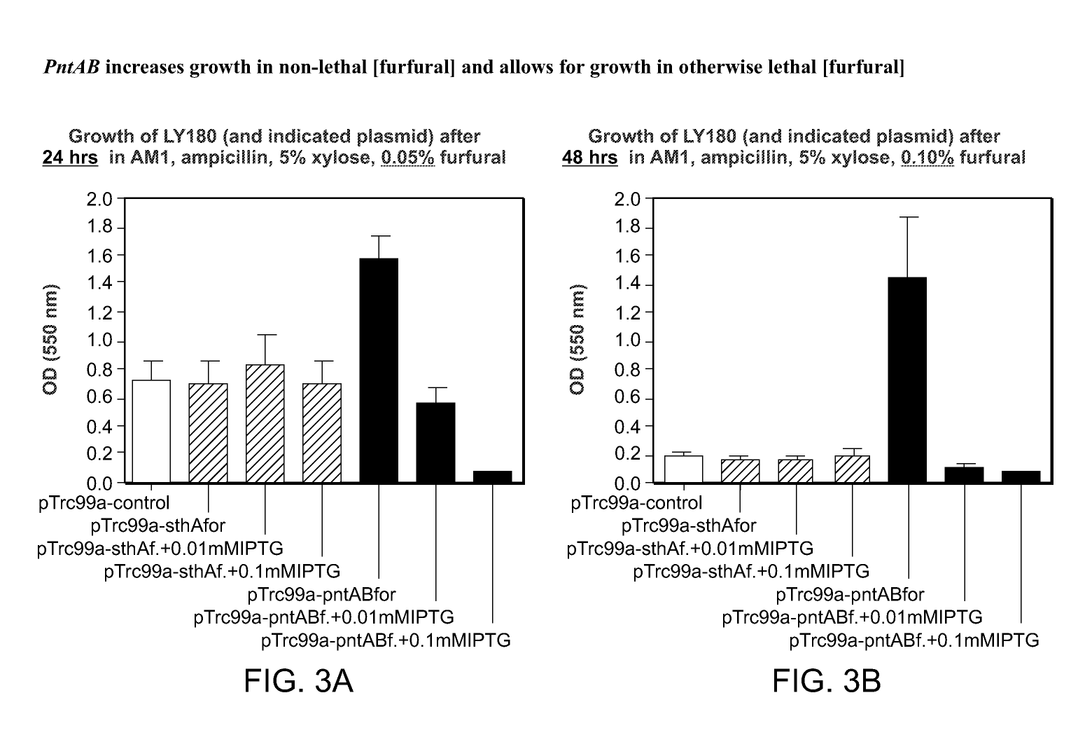

PntAB is a proton translocating transhydrogenase that is not known to function during fermentative growth but is potentially capable of increasing the pool of NADPH (52). In examples 2 and 3, the E. coli cytoplasmic transhydrogenase PntAB was cloned into pTrc99a and confirmed by sequencing. PntAB with native ribosomal binding site and rho dependent terminator was polymerase chain reaction (PCR) amplified from E. coli strain LY180 genomic DNA using primers with HindIII digestible ends. The PCR product and pTrc099a were HindIII digested, purified using a Qiagen Qiaprep Spin Miniprep Kit, and ligated together using T4 Quick DNA ligase. After transforming the resulting vector into TOP10F′, the plasmid was extracted and used to transform LY180. Orientation was verified by PCR analysis. The LY180 strains were modified with these plasmids as indicated in FIGS. 2-3. The empty vector served as a control. Inducer was added prior to inoculation. The growth of LY180 strains carrying plasmids having increasing expression of pntA and pntB genes was compared (FIG. 2A-2B). Optical density after 24 and 48 hour periods was plotted versus increasing furfural concentration. After 24 hours, at furfural concentration of 0.05%, the LY180 strain carrying the pTrc99a-pntAB forward plasmid showed an optical cell density measurement of over twice that of any of the other tested strains (FIG. 2A). After 48 hours, the LY180 strain carrying the pTrc99a-pntAB forward plasmid showed continued cell growth at a furfural concentration of 0.10% furfural while all the other strains had substantially stopped growing (FIG. 2B). These results indicate that selected pntA and pntB over-expression increases furfural tolerance.

### Example 3

The results of the growths of LY180 strains carrying plasmids having various levels of increased transhydrogenase sthA and pntAB gene expression were compared at 0.05% and 0.10% furfural concentrations after periods of 24 hours and 48 hours, respectively (FIG. 3A-B). After 24 hours, at a 0.05% furfural concentration, the LY180 strain carrying the pTrc99a-pntAB forward plasmid showed an optical cell density of approximately twice that of any of the other strains tested (FIG. 3A). After 48 hours, at a 0.10% furfural concentration, the LY 180 strain carrying the pTrc99a-pntAB forward plasmid showed an optical cell density of approximately 7 times that of any of the other strains tested and the other strains tested showed marginal growth, if any, at such furfural concentrations. Again, these results demonstrate that selected pntA and pntB over-expression increases furfural tolerance.

Leaky expression of pntAB from an uninduced plasmid partially restored growth in the presence of 1 g liter⁻¹ furfural (FIG. 3). Adding IPTG to express higher levels of this enzyme eliminated resistance to furfural and also inhibited the growth of cells in the absence of furfural. Based on these results, furfural inhibits growth by depleting the supply of NADPH needed for biosynthesis. The large requirement of NADPH for sulfate assimilation, 4 per cysteine equivalent, and the limited routes for NADPH production from xylose during fermentation made the production of sulfur amino acids most vulnerable to competition from furfural reductases for NADPH.

### Example 4

### Strains, Media, and Growth Conditions

Ethanologenic strains were maintained in AM1 mineral salts medium (22) supplemented with 20 g liter⁻¹ xylose for solid medium and 50 g liter⁻¹ xylose or higher for fermentation experiments. Strain E. coli LY180 (48, 60) is a derivative of KO11 and served as the starting point for this investigation. Note that E. coli W (ATCC 9637) is the parent of strain KO11, initially reported to be a derivative of E. coli B (29).

Fermentations were carried out as previously described (100 g liter⁻¹ xylose, 37° C., 150 rpm, pH 6.5) with the automatic addition of 2N KOH (23).

Furfural tolerance was examined by measuring growth in standing tubes with 4 mL total volume of AM1 and 50 g liter⁻¹ filter-sterilized xylose as described previously (48). Tubes were incubated at 37° C. and measured after 24 hours and 48 hours. Values reported are an average of 4 measurements.

### Strain Constructions

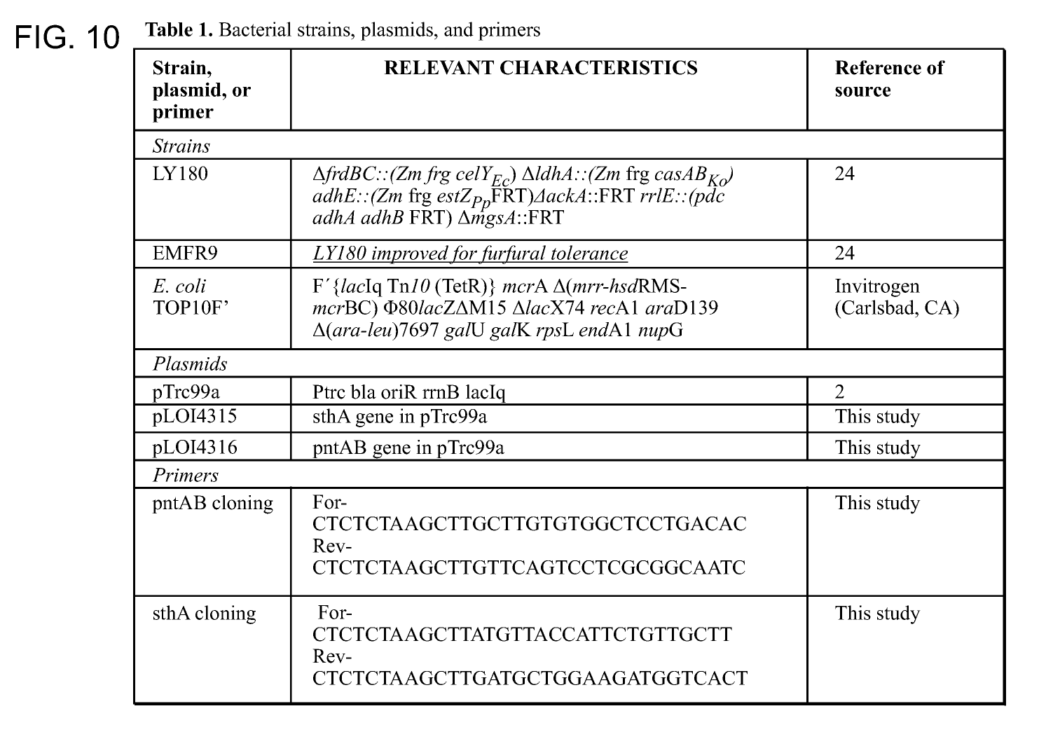

E. coli transhydrogenase genes were amplified (ribosomal-binding sites, coding regions, and a 200 bp terminator region) from strain LY180 genomic DNA using a BioRad iCycler (Hercules, Calif.) with primers that provided flanking HindIII sites (25). After digestion with HindIII, the product was ligated into HindIII digested pTrc99a (vector) and transformed into E. coli TOP10F′ (Carlsbad, Calif.). Plasmids were purified using a QiaPrep Spin Mini Prep Kit (Valencia, Calif.). Gene orientation was established by digestion with restriction enzymes and by polymerase chain reaction (Table 1 of FIG. 10).

### Microarray Analysis

Cultures were grown in small fermenters to a density of 670 mg dcw liter⁻¹. An initial sample was removed that served as a control. Furfural was immediately added from a 50 g liter⁻¹ aqueous stock (0.5 g liter⁻¹ final concentration) and incubation continued for 15 minutes prior to a second sampling. Samples were rapidly cooled in an ethanol-dry ice bath, harvested by centrifugation at 4° C., resuspended in Qiagen RNA Later and stored at −80° C. RNA was extracted using a Qiagen RNeasy Mini Kit, treated with DNase I and purified by phenol/chloroform extraction and ethanol precipitation. RNA was sent to NimbleGen (Madison, Wis.) for microarray comparisons using probes designed for E. coli K12. Each sample consisted of pooled material from four fermenters. The complete experiment was performed twice and the data was averaged (8 fermentors). Data was analyzed using ArrayStar (DNA Star, Madison, Wis.) and SimPheny software (Genomatica Inc., San Diego, Calif.). Expression ratios are presented as the average of the two pooled datasets. In an experiment adding only water as a control, 54 genes (>2% of transcriptome) were found to change by more than 2-fold after water addition. Only 8 of these were also affected by furfural addition, indicating that the transcriptional impact of the furfural delivery procedure was negligible relative to the effect of furfural.

### Network Component Analysis

Network Component Analysis (NCA) calculates transcription factor activity ratios from expression ratios and known regulatory connections and was performed as previously described (45, 46, 56). The connectivity file was updated according to Regulon DB and EcoCyc (4, 14). The regulon of the “stringent factor” was defined as previously described, by analysis of the 5-minute response to serine starvation via serine hydroxamate treatment during mid-log growth of BW25113 in MOPS glucose (12). Regulators with significantly altered regulatory activity were identified by comparison to a null distribution and using a P-value cutoff of 0.05.

### Transcriptomic Effect of Furfural

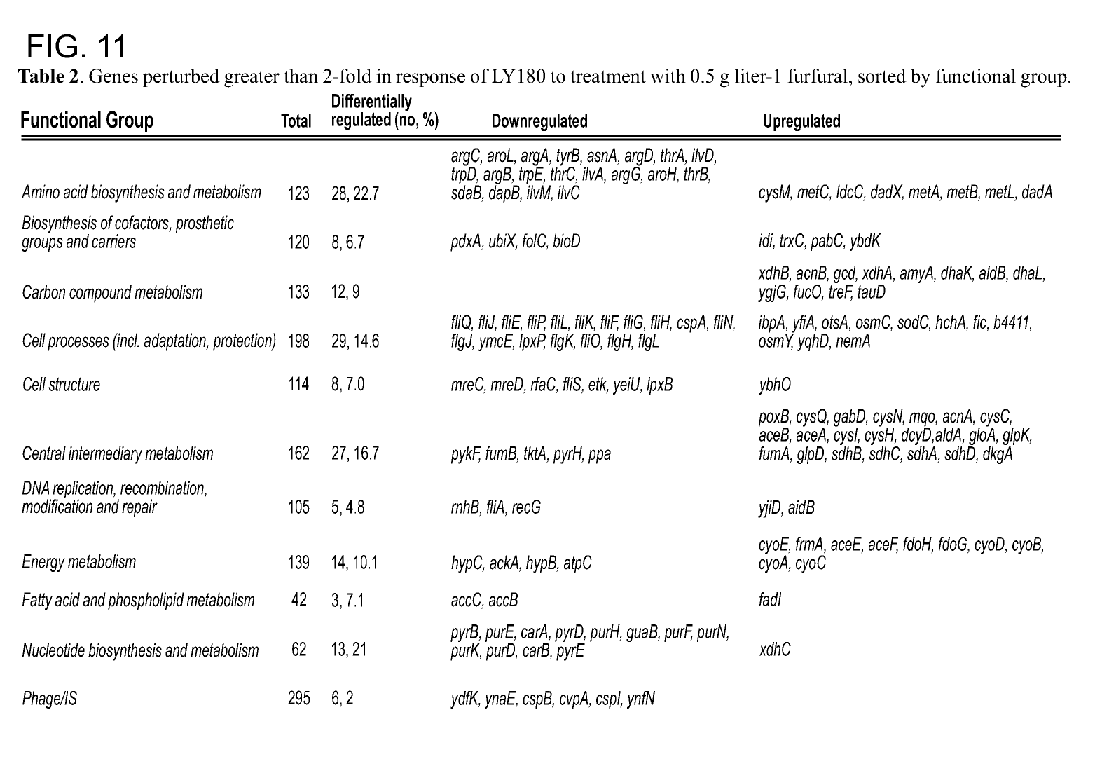

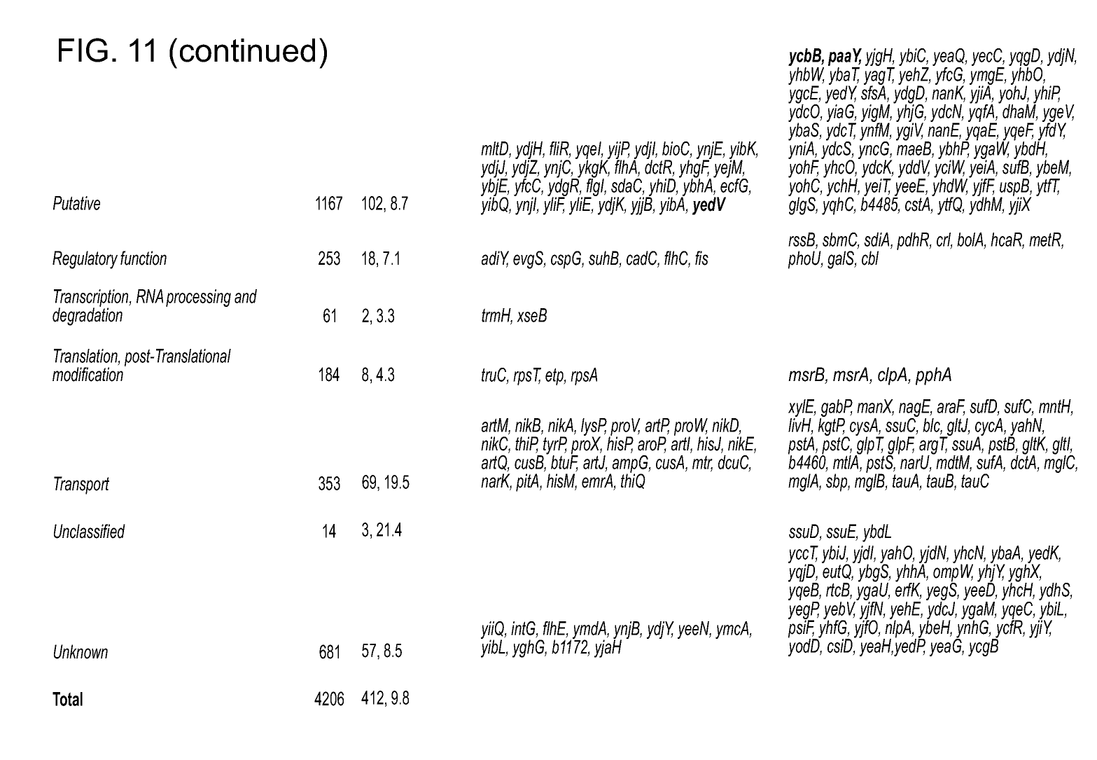

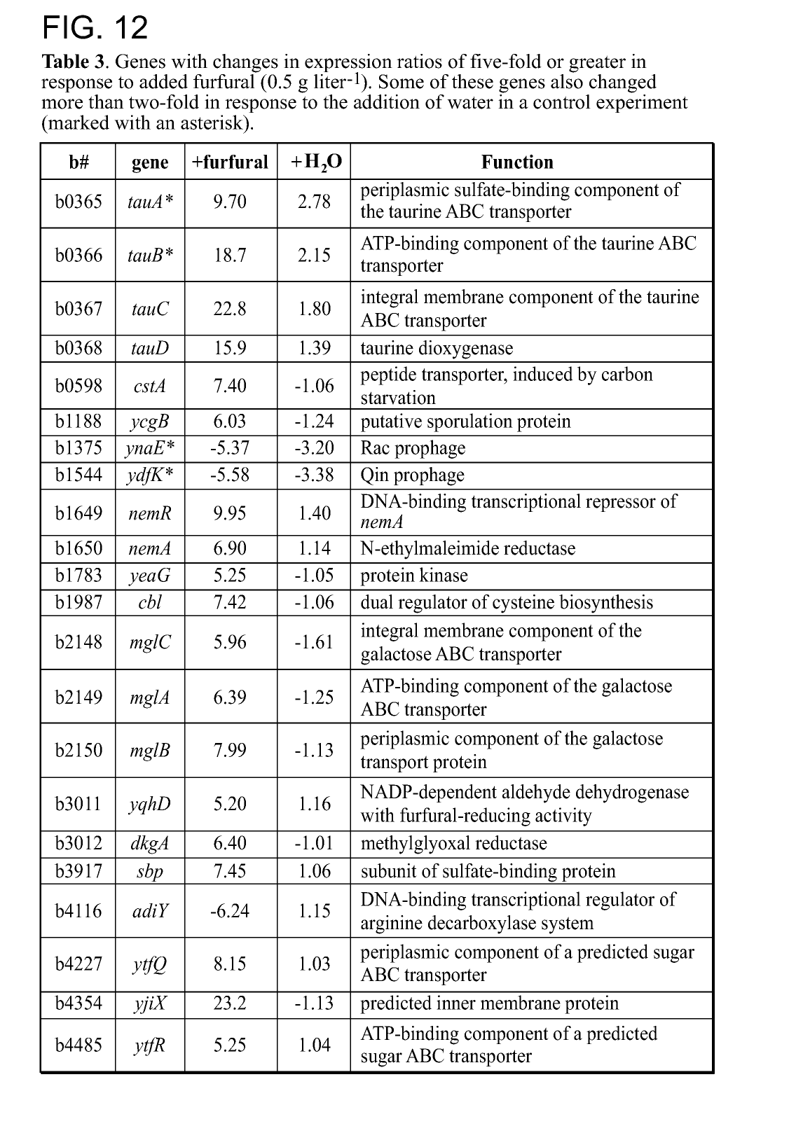

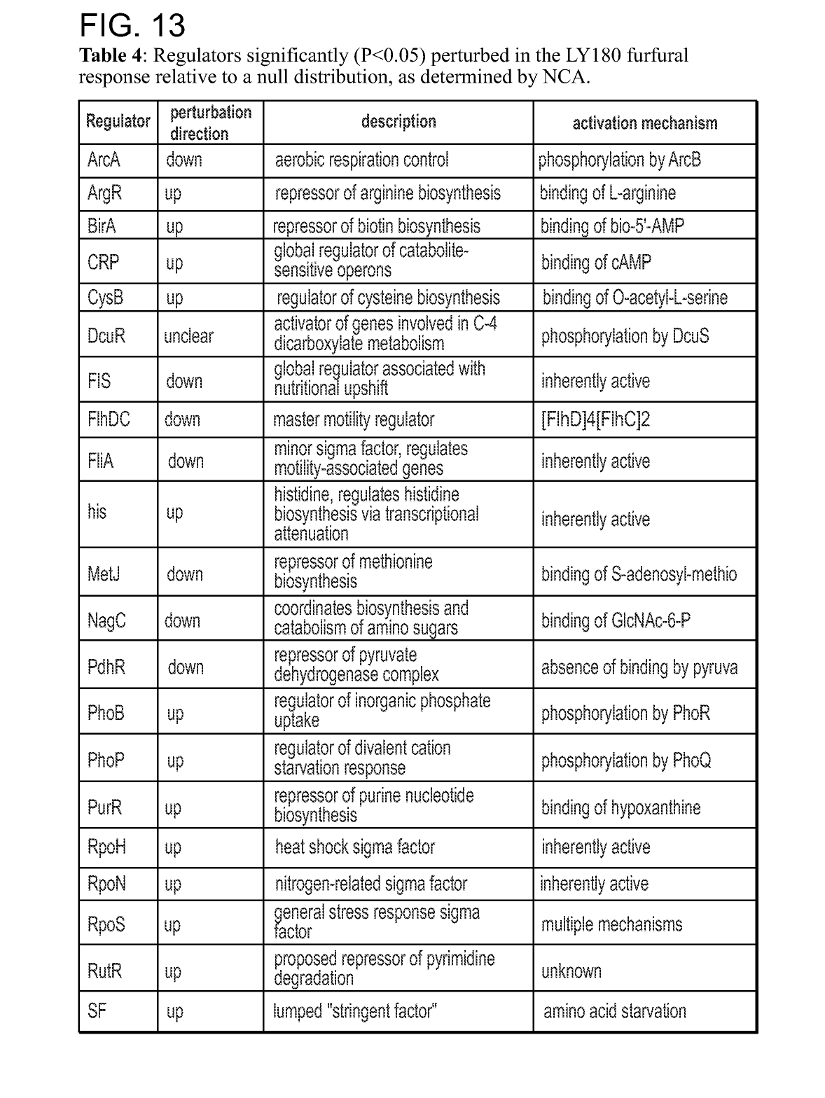

Message levels were compared in actively growing cells before and 15 min after the addition of 0.5 g liter⁻¹ furfural. A water control was also included for comparison. Expression of a small group of genes was found to be altered by more than five-fold in response to furfural (Table 3 of FIG. 12). Using a less stringent metric (two-fold or greater), expression levels for approximately 400 genes (10% of the transcriptome) were altered in response to furfural addition as compared to either the water control or the culture prior to furfural addition. The distribution of these altered genes varied widely among functional groups, providing useful insight into the mechanism of furfural's action (Table 2 of FIG. 11). In most functional groups, expression levels of less than 10% of the gene members were altered by 2-fold or greater. Groups with this low frequency of change included Cofactors, Carbon compounds, Regulatory, Macromolecular synthesis (Cell structure, DNA, Lipids, Transcription, and Translation), and others (Phage, Putative/IS, Regulatory, and Unclassified/Unknown). Expression levels for 10% to 20% of the member genes were altered in four groups (Cell processes, Central metabolism, Energy, and Transport). Most of the affected genes associated with central metabolism, energy, and transport increased in expression upon furfural addition. These changes provide an opportunity to scavenge and metabolize additional compounds that may be available and to increase carbon flow for energy production. Although many of the altered genes concerned with cell processes are involved in motility and chemotaxis, strain LY180 is non-motile (data not shown).

Consistent with a previous report (48), two oxidoreductases capable of catalyzing the NADPH-specific reduction of furfural, YqhD and DkgA, were up-regulated over 5-fold (mRNA) in the parent by the addition of furfural, as previously reported. These two genes were silenced in a furfural-resistant mutant (EMFR9), resulting in an increase in furfural tolerance.

Expression levels for over 20% of the member genes in two functional groups were altered by the addition of furfural, Amino acids and Nucleotides. In these groups, over ⅔ of the altered genes were reduced by 2-fold or greater upon the addition of furfural. Expression levels for individual genes affecting the biosynthesis of purines, pyrimidines, and every family of amino acids were reduced by 2-fold or greater upon the addition of furfural. A single gene in nucleotide metabolism and only 8 genes involved in amino acid metabolism exhibited a furfural-dependent increase in expression of at least 2 fold. Together, these changes agree with the general decrease in biosynthesis and growth observed upon the addition of furfural.

### Effect of Furfural on Regulatory Activity

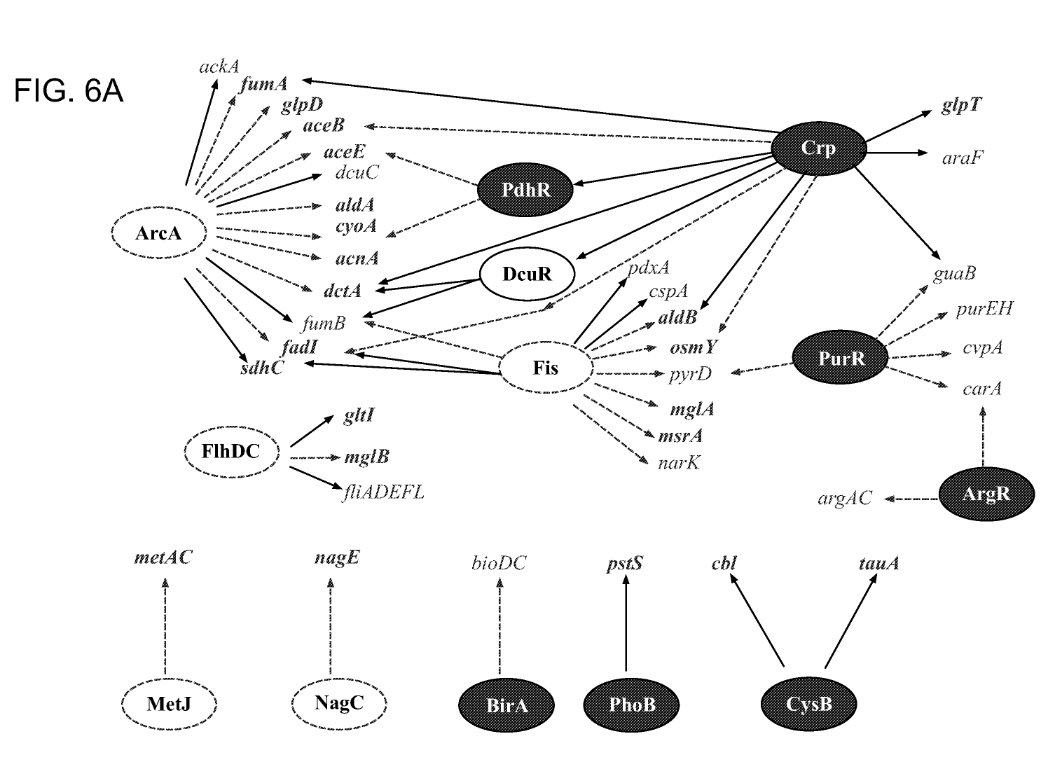

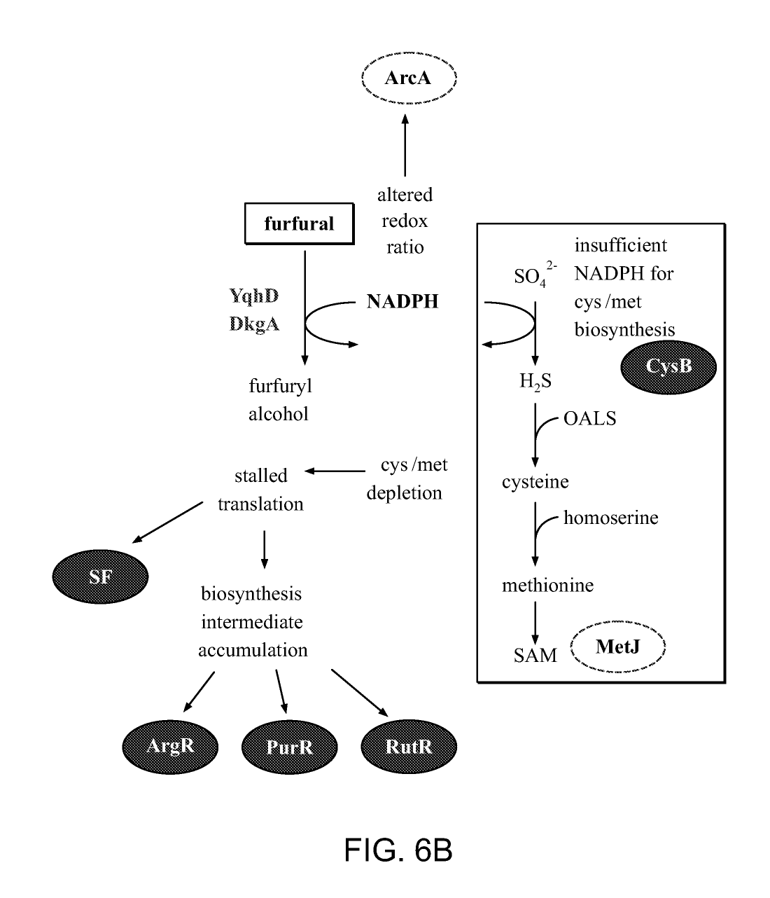

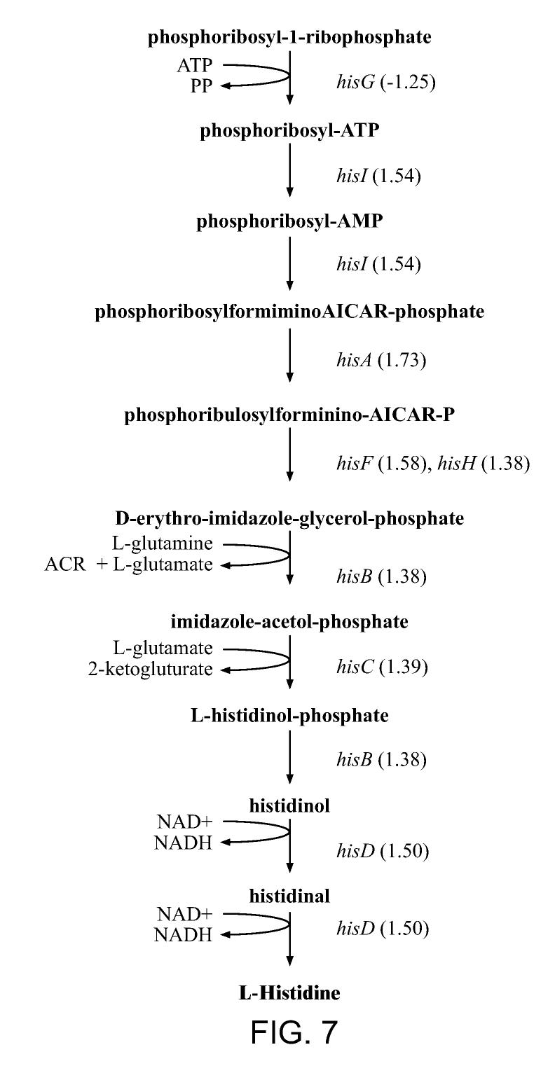

Network component analysis (NCA) was used to provide a global view of the cellular response to furfural. This analysis uses known regulatory network structure to identify regulators with perturbed activity from transcriptome data (46, 56). Of the 60 regulators included in this analysis, 22 were identified as having significantly altered expression after furfural challenge, where significance was assessed relative to a random network (FIG. 6A). Regulators of cysteine and methionine biosynthesis (CysB and MetJ) as well as repressors of amino acid (ArgR) and nucleotide biosynthesis (PurR) were significantly affected by furfural addition. The stringent factor, a collective indicator of the stringent response (diversion of resources away from growth during amino acid and carbon starvation) also shows activation consistent with stalled biosynthesis and an excess of many intermediates. Together, these results indicate that the pools of many amino acids and biosynthetic intermediates have been altered by furfural addition. The fact that expression of genes concerned with cysteine and methionine biosynthesis increased while expression of most other biosynthesis pathways declined is consistent with a depletion of cysteine and methionine pools as an early event resulting from a furfural challenge.

RpoS, a sigma factor that acts as a signal for general stress response, was also affected by the addition of furfural, indicating that the cell recognizes the presence of a stress-inducing agent. Since up to 10% of E. coli's genome is regulated in some fashion by RpoS (51, 59), it is difficult to determine a specific inhibitory response. Examples of RpoS-regulated genes with increased expression include poxB (conversion of pyruvate into acetate and CO₂) (64) and otsA (trehalose production during osmotic stress response) (55).

ArcA was significantly downregulated by NCA as evidenced by increased expression of numerous genes in central metabolism (aceB, aceE, sdhC, dctA, cyoA, fumA) and downregulation offumB, a gene typically known to be active during anaerobic conditions. The effect of glycerol supplementation on furfural tolerance was investigated. However, the addition of glycerol (1.0 to 20 g liter⁻¹) had no effect on furfural tolerance (data not included). Several other regulators, including fis and crp, were found to be significantly altered by NCA.

Histidine may also be limited by the addition of furfural. Genes (hisA, hisB, hisC, hisD, hisF, hisH, and hisI) under control of the His regulator (histidinyl-tRNA) were generally increased after the addition of furfural, although less than 2-fold (FIG. 7). The two terminal steps in histidine biosynthesis involve the reduction of NAD⁺ to NADH, a reaction that may be slowed by the high NADH/NAD⁺ ratio associated with fermentation.

Effects of Furfural on Expression of Genes Concerned with Sulfur Assimilation into Amino Acids

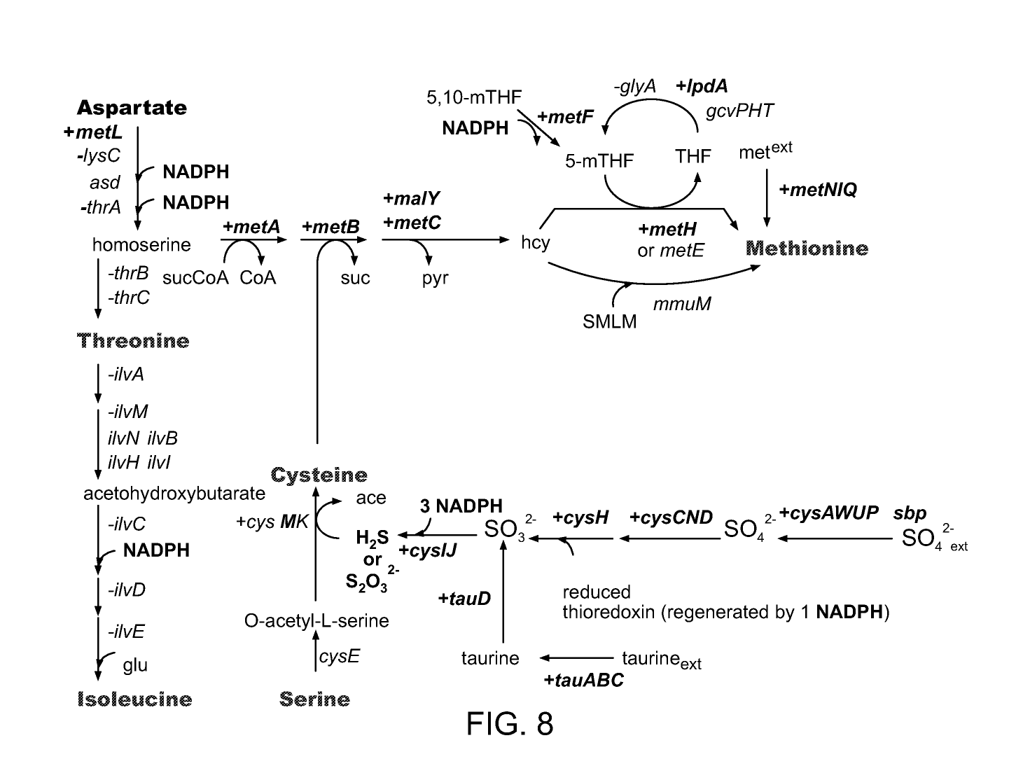

Many genes (cysC, cysH, cysl, cysM, cysN, cysQ, metA, metB, metC, metL, sbp, tauA, tauB, tauC and tauD) concerned with sulfur assimilation into cysteine, and methionine were increased by 2-fold or greater. These are scattered within several functional groups (Table 2 of FIG. 11): Amino acid, Central metabolism, Regulation, and Transport. Many additional genes involved in sulfur assimilation were also up-regulated less than 2-fold and are included to demonstrate the broad furfural response (FIG. 8). Sulfur is supplied as sulfate in AM1 medium and must be reduced to the level of hydrogen sulfide for incorporation, an energy intensive reaction requiring 4 molecules of NADPH. The furfural-induced increase in expression of these genes is in sharp contrast to the decreases observed for many other genes concerned with the biosynthesis of amino acids, purines, and pyrimidines (Table 2 of FIG. 11). Expression of the taurine transport genes (tauABCD; alternative source of sulfur), the sulfate-binding transport protein (sbp), and the transcriptional activator of many cysteine biosynthetic genes (cbl) were increased by more than 5-fold in response to added furfural (Table 3 of FIG. 12). Together, these results demonstrate that the addition of furfural results in an intracellular deficit in sulfur-containing amino acids (cysteine and methionine) which is associated with the high NADPH requirement in this pathway.

### Effect of Amino Acid Supplements on Furfural Tolerance

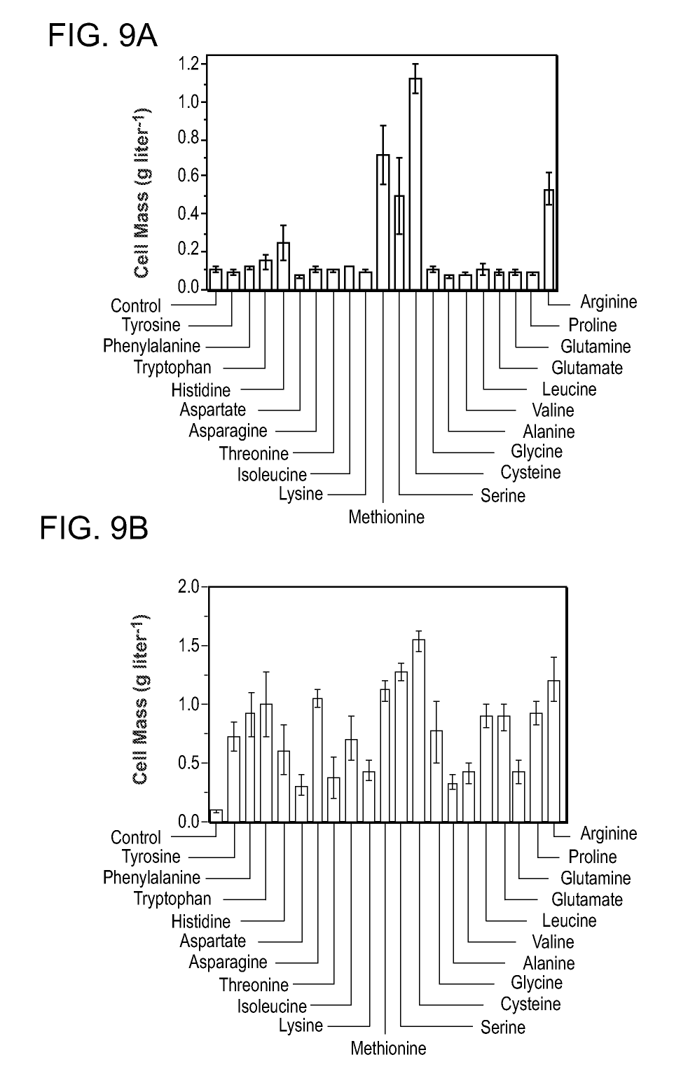

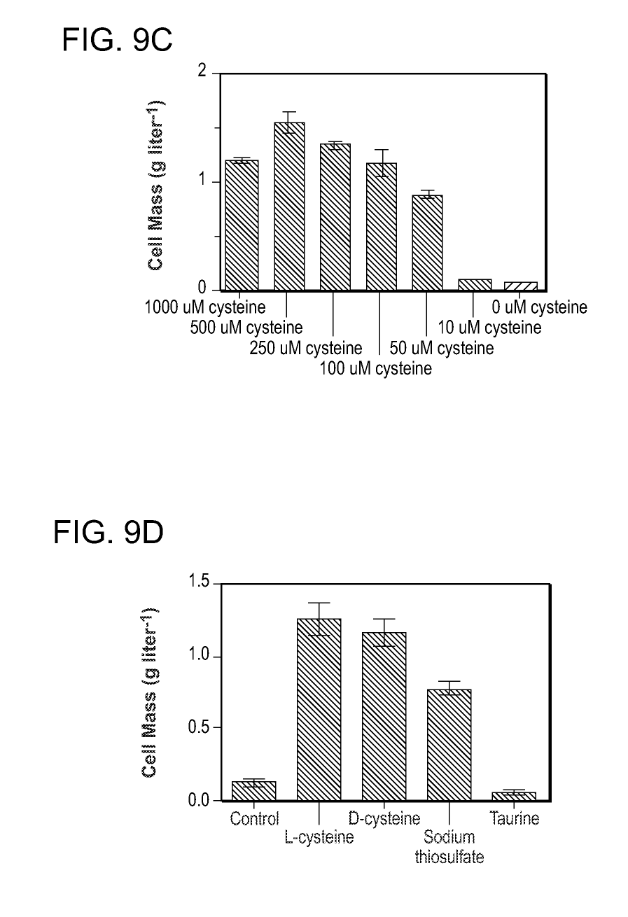

All 20 amino acids were individually tested for their ability to improve the growth of LY180 in AM1 mineral salts medium (FIG. 9A). A concentration of 0.1 mM was selected roughly based on the cellular content of individual amino acids (50). Only five amino acids improved furfural resistance when supplied at this low concentration: cysteine>methionine>serine, arginine>histidine. The two sulfur amino acids were clearly the most beneficial for furfural resistance. When supplied at a 5-fold higher concentration (0.5 mM), all amino acids were beneficial to some degree (FIG. 9B). However, cysteine remained the most effective followed by serine, methionine, and arginine. A cysteine concentration of 0.05 mM allowed LY180 to grow to a density of 0.8 g liter⁻¹ in the presence of 1 g liter⁻¹ furfural, approximately equal to the total cellular sulfur (FIG. 4C). No measurable improvement in furfural resistance was observed with 0.01 mM cysteine.

Protective concentrations of cysteine (0.05 mM) were 200-fold lower than that of 1 g liter⁻¹ furfural (10 mM). Furfural in mineral salts medium is readily quantified by its characteristic spectrum (Martinez et al. 2000) and remained unchanged during 48 incubation at 37° C., consistent with minimal chemical reactivity.

Most genes concerned with histidine biosynthesis increased in response to furfural addition, although less than 2-fold (FIG. 7). De novo biosynthesis of histidine during fermentation is constrained by the high NADH/NAD⁺ ratio during anaerobic growth and the requirement for further reduction of NAD⁺ in the two terminal steps of biosynthesis. Similarly, the first committed step in serine biosynthesis also involves the reduction of NAD⁺ and is hindered during fermentation. Increasing serine increases the efficiency of incorporating reduced sulfur from H₂S into cysteine. Genes concerned with arginine biosynthesis (argA, argB, argC, argD, carA, carB, and argG) were generally lowered by more than 2-fold upon the addition of furfural. The expression level of speA encoding arginine decarboxylase was increased by the addition of furfural. The degradation of arginine provides useful intermediates and co-factors for biosynthesis.

### Effect of Alternative Sulfur Sources on Furfural Tolerance

The addition of furfural inhibited growth and increased the transcription of genes concerned with sulfur assimilation. Genes involved in the uptake and incorporation of the alternative sulfur compound, taurine (tauABC and tauD), were among the 10 genes with the largest increases in expression (Table 3 of FIG. 12). The tau genes are typically expressed only during sulfur starvation (57). Since cysteine was effective in relieving furfural inhibition, the increased expression of these genes results from a reduction in the pool of sulfur amino acids by furfural. Furfural inhibits sulfur amino acid biosynthesis either by limiting the availability of reduced sulfur (H₂S) from sulfate or by inhibiting the incorporation of reduced sulfur into cysteine.

During growth in AM1 medium containing 1 g liter⁻¹ furfural, the effects of alternative sulfur sources (L-cysteine, D-cysteine, taurine, and sodium thiosulfate) that enter metabolism at different levels of reduction (FIG. 9D) was compared. Note that 4 NADPH molecules and two reductase enzymes (CysH and CysIJ) are required to fully reduce sulfate prior to assimilation into cysteine. L-cysteine, D-cysteine and thiosulfate bypass both reductase enzymes and all were effective at relieving furfural inhibition. D-cysteine cannot be incorporated directly and is first catabolized to H₂S (49). Thiosulfate also serves as a source of reduced sulfur for incorporation by CysM (47). Taurine is catabolized to sulfite in the cytoplasm and must be reduced by sulfite reductase (CysIJ and 3 NADPH molecules) prior to assimilation into cysteine (54). Unlike cysteine and thiosulfate, taurine was not effective in preventing the inhibition of growth by 1 g liter⁻¹ furfural.

These results with alternative sulfur sources indicate that furfural acts to inhibit growth by limiting the production of reduced H₂S from sulfate rather than by inhibiting the incorporation of reduced sulfur into cysteine. With taurine as a sulfur source, furfural must act at the level of sulfite reductase (CysIJ), which has a higher Km for NADPH (80 μM) (53) than the furfural reductase YqhD (8 μM) (48). With sulfate as a sulfur source, further effects of furfural at earlier steps in sulfur assimilation cannot be excluded.

### Example 5

### Increase in 5-Hydroxymethyl Furfural Resistance in Ethanol-Producing Bacteria

The ability of a biocatalyst to tolerate furan inhibitors present in hemicellulose hydrolysates is important for the production of renewable chemicals. This example demonstrates that EMFR9, a furfural-tolerant mutant of ethanologenic E. coli LY180, has also acquired tolerance to 5-hydroxymethyl furfural (5-HMF). Furan tolerance results from lower expression of yqhD and dkgA, two furan reductases with a low Kₘ for NADPH. Furan tolerance was also increased by adding plasmids encoding a NADPH/NADH transhydrogenase (pntAB).

### Materials and Methods

### Strains, Media, and Growth Conditions

Strains and plasmids used in this study have been previously described (70 and 23). These include LY180 (an ethanologenic derivative of E. coli), EMFR9 (furfural-tolerant derivative of LY180), LY180ΔyqhD, LY180ΔdkgA, LY180ΔyqhD ΔdkgA, pLOI4301 containing yqhD. Plasmids pLOI4303 containing dkgA (48), and pLOI4316 containing pntAB (70) were also used. Cultures were grown at 37° C. in AM1 minimal media (23) containing 20 g 1⁻¹ xylose (solid medium), 50 g l⁻¹ xylose (Bioscreen C growth analyzer and tube cultures), or 100 g 1⁻¹ (pH-controlled fermentations).

Tolerance to HMF was tested using 13×100 mm closed tubes containing 4 ml AM1 and 5-HMF as indicated. When appropriate, antibiotics were included for plasmid maintenance. Tubes were inoculated to an initial density of 0.05 OD₅₅₀ₙₘ. Growth was measured after incubation (60 rpm) for 48 h using a Spectronic 20D+ spectrophotometer (Thermo, Waltham, Mass.). To examine the effects of pntAB on furan tolerance, a multiwall plate containing 400 μl of AM1 (and 5-HMF or furfural) per well was inoculated as above. OD_{(420-580nm bandwidth)} was measured for 72 h using a Bioscreen C growth analyzer (Oy Growth Curves, Helsinki, Finland).

For fermentation experiments, seed cultures of LY180 and EMFR9 were grown overnight in small fermentors (37° C., 200 rpm) containing 350 ml of AM1 medium. Broth was maintained at pH 6.5 by the automatic addition of 2 N KOH. Upon reaching mid-log phase, experimental fermenters were inoculated to an initial cell density of 0.1 OD₅₅₀ₙₘ. (33 mg dry cell weight 1⁻¹). Cell mass (OD₅₅₀ₙₘ) and furan levels were monitored at 12-h intervals as described previously (20).

Furan reduction in vivo was measured using pH-controlled fermenters. Furans were added when the cultures reached approximately 1 OD₅₅₀ₙₘ using a 10% w/v stock solution. Cell mass and 5-HMF were measured after 0, 15, 30, and 60 minutes.

### In Vitro Furan Reduction

Culture tubes (13×100 mm) containing AM1 and 0.1 mM IPTG were inoculated to 0.05 OD₅₅₀ₙₘ and incubated at 37° C. These were harvested at a density of 1-2 OD₅₅₀ₙₘ. Cell pellets were washed once with 100 mM potassium phosphate buffer (pH 7.0), and resuspended in buffer at a density of 10 OD₅₅₀ₙₘ. Samples (1 ml) were added to 2-ml tubes containing Lysing Matrix B and disrupted (20 s) using a FastPrep-24 (MP Biomedicals, Solon, Ohio). Furan-dependent oxidation of NADPH was measured at 340 nm using a DU 800 spectrophotometer (Beckman Coulter, Fullerton, Calif.). Reactions (200 μl total volume; 37° C.) contained 50 μL crude extract, 0.2 mM NADPH, and 20 mM 5-HMF. Protein was measured using the BCA assay (Thermo Scientific, Rockford, Ill.).

### Statistical Analysis

Data are presented as an average±SD (n≧3). Statistical comparisons (2-tailed student-t test) were made using Graphpad Prism software (La Jolla, Calif.).

### Results

### Strain EMFR9 Exhibits Increased Tolerance to 5-HMF

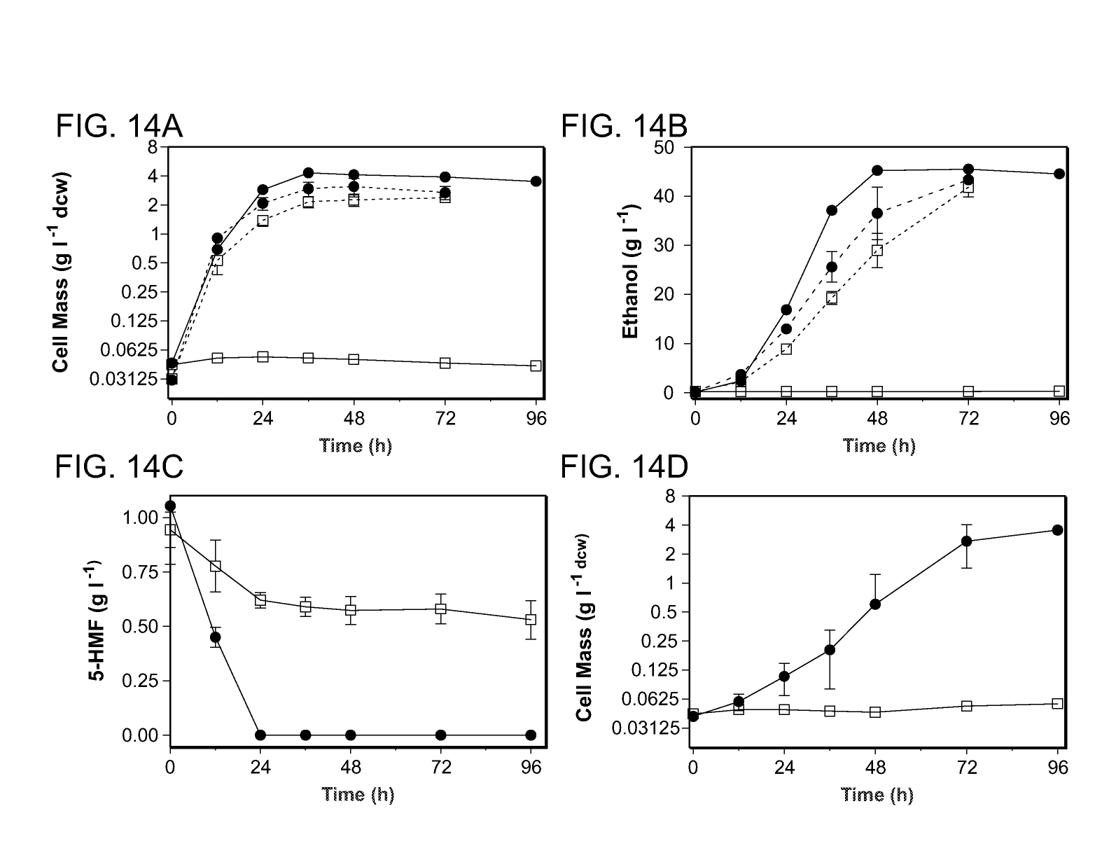

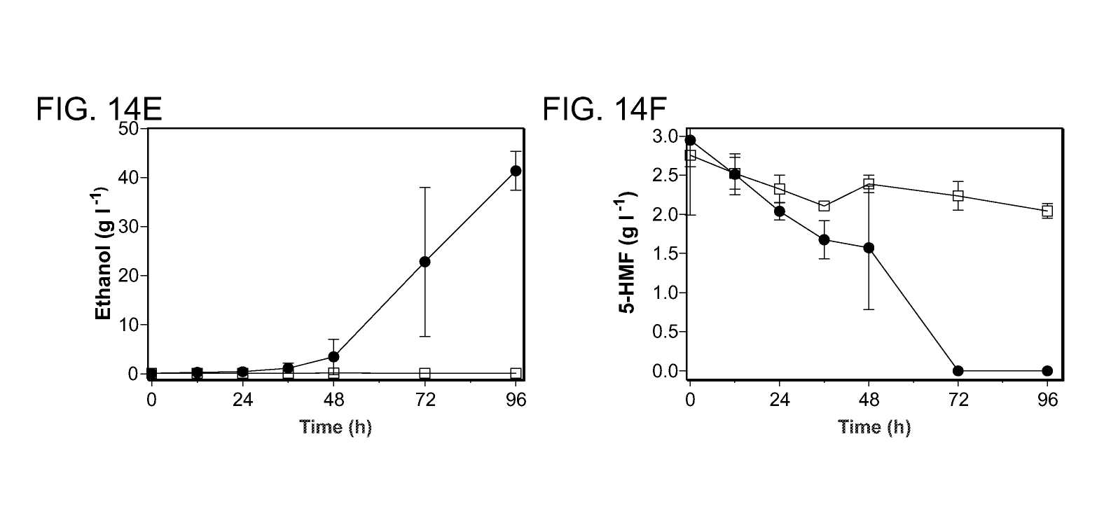

Mutations present in the furfural-resistant mutant, EMFR9, also increased resistance to 5-HMF (FIG. 14). At 1.0 g l⁻¹ 5-HMF, growth and ethanol production by EMFR9 were equal to that of LY180 (parent) in the absence of 5-HMF (FIG. 14A, 14B). 5-HMF was rapidly metabolized by EMFR9 during the initial 24 h of fermentation with no detrimental effect on cell yield or ethanol yield. The growth of LY180 was completely inhibited by 1.0 g l⁻¹ 5-HMF, although 5-HMF levels declined slowly during incubation (FIG. 14A, 14B, and 14C). No decline was observed without inoculation (data not shown) confirming that this is the result of metabolic activity.

With EMFR9, ethanol production and growth were slowed by inclusion of 2.5 g 5-HMF l⁻¹ but proceeded to completion after 96 h (FIG. 14D, 14E, and 14F). Cell and ethanol yields with this higher level of 5-HMF were comparable to LY180 without 5-HMF. The level of 5-HMF declined rapidly and completely with EMFR9. With LY180, metabolism of 5-HMF was slow and incomplete (FIG. 14F).

### Effects of YqhD and DkgA on 5-HMF Tolerance

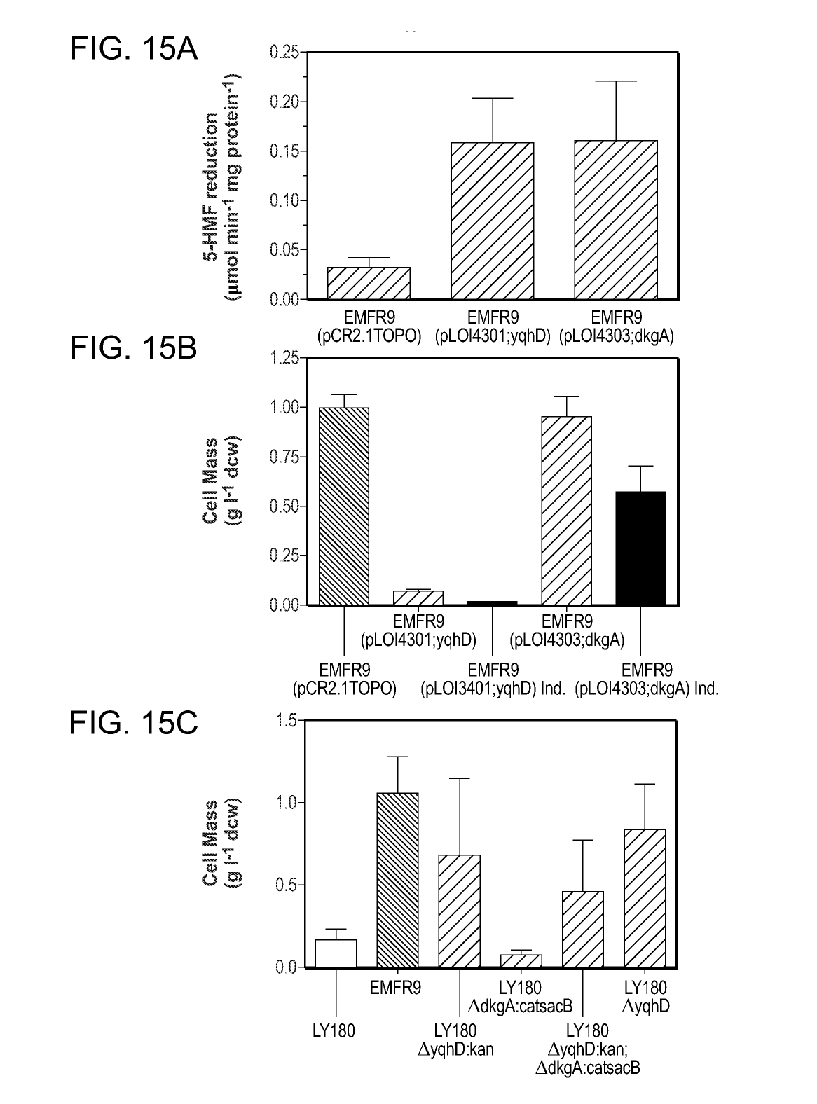

Furfural tolerance in EMFR9 was previously demonstrated to result from the silencing of two NADPH-dependent oxidoreductases, YqhD and DkgA (23). Genes encoding these activities were cloned into pCR2.1 TOPO, transformed into EMFR9, and induced with 0.1 mM IPTG. Cells were harvested, disrupted, and tested for 5-HMF reductase activity (FIG. 15A). Expression of yqhD and dkgA individually from plasmids resulted in a 5-fold increase in the rate of 5-HMF-dependent oxidation of NADPH, confirming that YqhD and DkgA use 5-HMF as a substrate.

The individual expression of yqhD and dkgA from plasmids decreased the tolerance of EMFR9 to 5-HMF (FIG. 15B). Addition of kanamycin (12.5 mg l⁻¹) for plasmid maintenance decreased 5-HMF tolerance in all strains, requiring the use of a lower concentration of 5-HMF (1.0 g l⁻¹) in this experiment. Plasmid pCR2.1 is leaky for the expression of cloned genes in the absence of IPTG (71). Even uninduced expression of yqhD was sufficient to restore the sensitivity of EMFR9 to 5-HMF. Growth inhibition by 5-HMF was further increased by yqhD induction. Expression of dkgA was less effective and required induction to restore 5-HMF sensitivity in EMFR9. Differences in effectiveness between these two oxidoreductases are consistent with the lower apparent Kₘ of YqhD (8 μM) for NADPH compared to 23 μM for DkgA (23).

Deletion of yqhD from LY180 increased tolerance to 2.5 g 1⁻¹ 5-HMF (FIG. 15C). Deletions in which markers remained in the chromosome were less effective but confirmed that the inactivation of yqhD was beneficial for 5-HMF tolerance in all cases.

### Increasing the Availability of NADPH Increased 5-HMF Tolerance

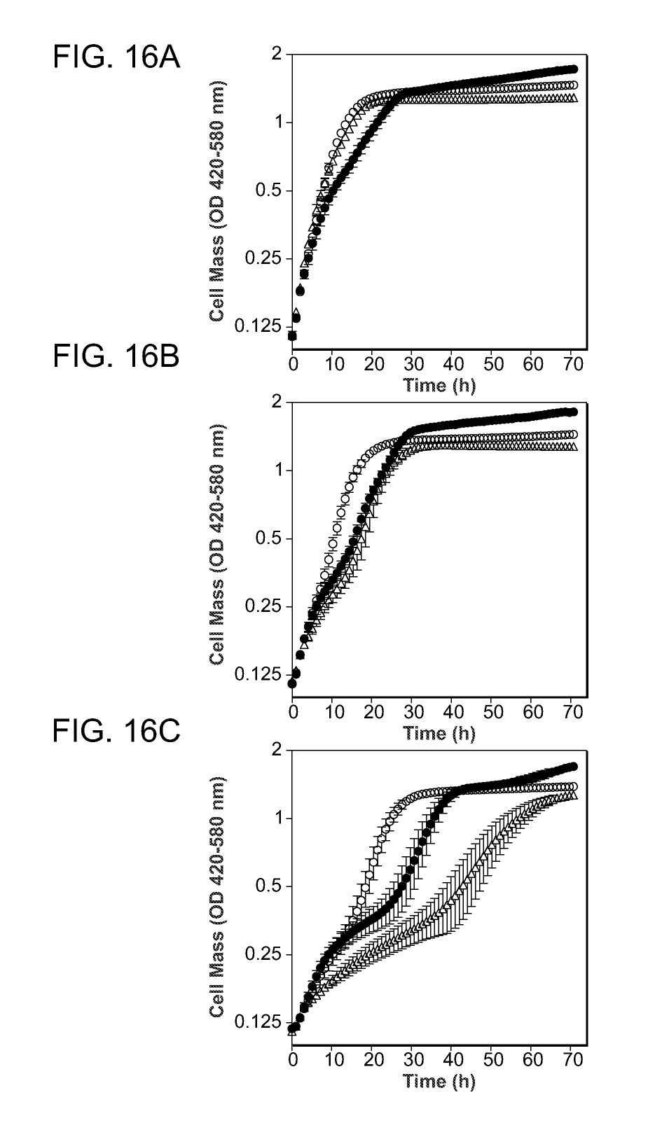

The proton-translocating transhydrogenase pntAB (68) was over-expressed in LY180 (FIG. 16) to increase the availability of NADPH. In the absence of inhibitor (FIG. 16A), both LY180 with the vector (control) and LY180 (pTrc99a pntAB) grew at the same rate. Induction of LY180 (pTrc99a-pntA) with IPTG (0.01 mM) was detrimental in the absence of 5-HMF. Uninduced LY180 (pTrc099apntAB), however, grew more rapidly than the vector control (FIGS. 16B and 16C) in the presence of 5-HMF (0.9 g l⁻¹ and 1.8 g l⁻¹). A similar benefit of pntAB was observed previously with furfural (23). Thus the inhibition of growth by both furans appears to result from furan reduction, depleting the pool of NADPH required for biosynthesis. In addition, over-expression of pntAB led to an increase in overall growth after 72 h, even in the absence of furfural, indicating that biosynthesis may be limited by NADPH under these conditions.

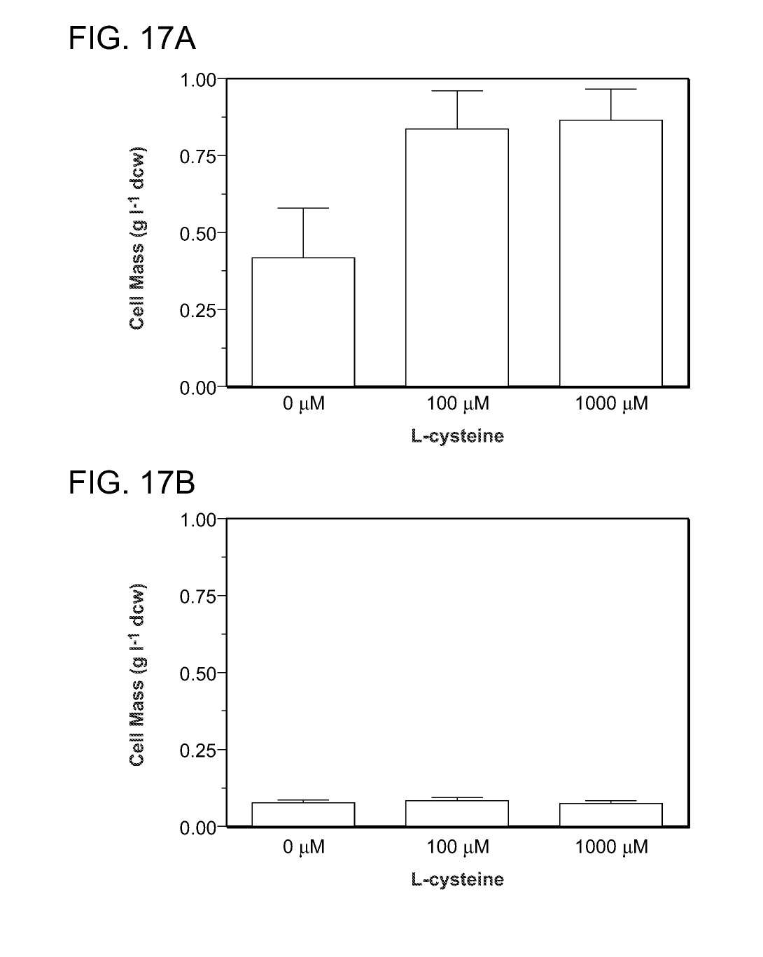

Sulfur assimilation and cysteine biosynthesis have a particularly high requirement for NADPH. Supplementing with cysteine was previously shown to increase furfural tolerance in E. coli LY180 (70) but was found to be of less benefit for 5-HMF tolerance (FIG. 17). Growth of LY180 was partially inhibited by 1 g l⁻¹ 5-HMF and completely restored by supplementing with 100 μM cysteine (FIG. 17A). Growth in the presence of 2.5 g l⁻¹ 5-HMF was not restored by 100 μM or 1000 μM cysteine (FIG. 17B). Unlike furfural, cysteine supplements did not increase the MIC for 5-HMF.

### REFERENCES

1. Almeida, J. R. M., T. Modig, A. Petersson, B. Hahn-Hagerdal, G. Liden, and M. F. Gorwa-Grauslund. 2007. Increased tolerance and conversion of inhibitors in lignocellulosic hydrolysates by Saccharomyces cerevisiae. J. Chem. Technol. Biotechnol. 82:340-349.
2. Almeida, J. R. M. A. Roder, T. Modig, B. Laddan, G. Lida', and M. F. Gorwa-Grauslund. 2008. NADH- vs. NADPH-coupled reduction of 5-hydroxymethyl furfural (HMF) and its implications on product distribution in Saccharomyces cerevisiae. Appl. Microbiol. Biotechnol. 78:939-945.
3. Barciszewski, J., G. E. Siboska, B. O. Pedersen, B. F. C. Clark, and S. I. S. Ratten. 1997. A mechanism for the in vivo formation of N-6-furfuryladenine, kinetin, as a secondary oxidative damage product of DNA. FEBS letters. 414:457-460.
4. Boopathy, R., H. Bokang, and L. Daniels. 1993. Biotransformation of furfural and 5-hydroxymethyl furfural by enteric bacteria. J. Indust. Microbiol. 11:147-150.
5. Datsenko, K. A., and B. L. Wanner. 2000. One-step inactivation of chromosomal genes in Escherichia coli K-12 using PCR products. PNAS. 97:6640-6645.
6. Gorsich, S. W., B. S. Dien, N. N. Nichols, P. J. Slinger, Z. L. Liu, and C. D. Skory. 2006. Tolerance to furfural-induced stress is associated with pentose phosphate pathway genes ZWF1, GND1, RPE1, and TKL1, in Saccharomyces cerevisiae. Appl. Microbiol. Biotechnol. 71:339-349.
7. Gutiérrez, T., L. O. Ingram, and J. F. Preston. 2006. Purification and characterization of a furfural reductase (FFR) from Escherichia coli strain LY01-An enzyme important in the detoxification of furfural during ethanol production. J. Bacteriol. 121:154-164.
8. Habrych, M., S. Rodriguez, and J. Stewart. 2002. Purification and identification of an Escherichia coli Beta-Keto Ester Reductase as 2,5-diketo-D-gluconate reductase YqhE. Biotechnol. Prog. 18:257-261.
9. Hahn-Hägerdal, B., M. Galbe, M. F. Gorwa-Grauslund, G. Liden, and G. Zacchi. 2006. Bio-ethanol—the fuel of tomorrow from the residues of today. Trends Biotechnol. 24:549-556.
10. Horvath, I. S., M. J. Taherzadeh, C. Niklasson, and G. Liden. 2001. Effects of furfural on anaerobic continuous cultivation of Saccharomyces cerevisiae. Biotechnol. Bioeng. 75:540-549.
11. Hristozova, Ts., A. Angelov, B. Tzvetkova, D. Paskaleva, V. Gotcheva, S. Gargova, and K. Pavlova. 2006. Effect of furfural on carbon metabolism key enzymes of lactose-assimilating yeasts. Enzyme Microbiol. Technol. 39:1108-1112.
12. Jantama, K., X. Zhang, J. C. Moore, K. T. Shanmugam, S. A. Svoronos, and L. O. Ingram. 2008. Eliminating side products and increasing succinate yields in engineered strains of Escherichia coli C. Biotech. Bioeng. 30:881-893.
13. Jarboe, L. R., T. B. Grabar, L. P. Yomano, K. T. Shanmugan, and L. O. Ingram. 2007. Development of ethanologenic bacteria. Adv. Biochem. Engin/Biotechnol. 108:237-261.
14. Jeudy, S., V. Monchois, C. Maza, J M Clayerie, C. Abergel. 2006. Crystal structure of Escherichia coli DkgA, a broad-specificity aldo-keto reductase. Proteins. 62:302-307.
15. Karp, P. D., I. M. Keseler, A. Shearer, M. Latendresse, M. Krummenacker, S. M. Paley, I. Paulsen, J. Collado-Vides, S. Gama-Castro, M. Peralta-Gil, A. Santos-Zavaleta, M. I. Penaloza-Spinola, C. Bonavides-Martinez, and J. Ingraham. 2007. Multidimensional annotation of the Escherichia coli K12 genome. Nucleic Acids Res. 35:7577-7590.
16. Katahira, S., A. Mizuike, H. Fukuda, and A. Kondo. 2006. Ethanol fermentation from lignocellulosic hydrolysate by a recombinant xylose and cellooligosaccharide-assimilating yeast strain. Appl. Microbiol. Biotechnol. 72:1136-1143.
17. Khan, Q. A., F. A. Shamsi, and S. M. Hadi (1995) Mutagenicity of furfural in plasmid DNA. Cancer Lett. 89:95-99.
18. Ko, J., I. Kim, S. Yoo, B. Min, K. Kim, and C. Park. 2005. Conversion of methylglyoxal to acetol by Escherichia coli aldo-keto reductases. J. Bacteriol. 187:5782-5789.
19. Martin, C., M. Marcet, O. Almazan, and L. J. Jonsson. 2007. Adaptation of a recombinant xylose-utilizing Saccharomyces cerevisiae strain to a sugarcane bagasse hydrolysate with high content of fermentation inhibitors. Bioresour. Technol. 98:1767-1773.
20. Martinez, A., M. E. Rodriguez, S. W. York, J. F. Preston, and L. O. Ingram. 2000. Use of UV absorbance to monitor furans in dilute acid hydrolysates of biomass. Biotechnol Prog. 16:637-641.
21. Martinez, A., M. E. Rodriguez, M. L. Wells, S. W. York, J. F. Preston, and L. O. Ingram. 2001. Detoxification of dilute acid hydrolysates of lignocellulose with lime. Biotechnol. Prog. 17:287-293.
22. Martinez, A., M. E. Rodriguez, S. W. York, J. F. Preston and L. O. Ingram. 2000. Effects of Ca(OH)₂ treatments (“overliming”) on the composition and toxicity of bagasse hemicellulose hydrolysates. Biotechnol. Bioengin. 69(5): 526-536.
23. Martinez, A., T. B. Grabar, K. T. Shanmugam, L. P. Yomano, S. W. York, and L. O. Ingram. 2007. Low salt medium for lactate and ethanol production by recombinant Escherichia coli B. Biotechnol. Lett. 29:397-404.
24. McCue, L. A., W. Thompson, C. S. Carmack, M. P. Ryan, J. S. Liu, V. Derbyshire, and C. E. Lawrence. 2001. Phylogenetic footprinting of transcription factor binding sites in proteobacterial genomes. Nucleic Acids Res. 29:774-782.
25. Miller, J.H.1992. A short course in bacterial genetics. CSHL Press. Plainview, N.Y.
26. Modig, T., G. Liden, and M. J. Taherzadeh. 2002. Inhibition effects of furfural on alcohol dehydrogenase, aldehyde dehydrogenase, and pyruvate dehydrogenase. Biochem. J. 363:769-776.
27. Nakamura, C. E., and Whited G M. 2003. Metabolic engineering for the microbial production of 1,3-propandiol. Current Opinion in Biotechnology. 14:454-459.
28. Nilsson, A., M. F. Gorwa-Grauslund, B. Hahn-Hagerdal, and G. Liden. 2005. Cofactor dependence in furan reduction by Saccharomyces cerevisiae in fermentation of acid-hydrolyzed lignocellulose. Appl. Environ. Microbiol. 71:7866-7871
29. Ohta, K., D. S. Beall, K. T. Shanmugam, and L. O. Ingram. 1991. Genetic improvement of Escherichia coli for ethanol production: chromosomal integration of Zymomonas mobilis genes encoding pyruvate decarboxylase and alcohol dehydrogenase II. Appl. Environ. Microbiol. 57:893-900.
30. Palmqvist, E., and B. Hahn-Hagerdal. 2000. Fermentation of lignocellulosic hydrolysates. I: inhibition and detoxification. Bioresour. Technol. 74:17-24.
31. Palmqvist, E., and B. Hahn-Hagerdal. 2000. Fermentation of lignocellulosic hydrolysates. II: inhibitors and mechanisms of inhibition. Bioresour. Technol. 74:25-33.
32. Pérez, J. M., F. A. Arenas, G. A. Pradenas, J. M. Sandoval, and C. C. Vásquez. 2008. Escherichia coli YqhD exhibits aldehyde reductase activity and protects from the harmful effect of lipid peroxidation-derived aldehydes. J. Biol. Chem. 283:7346-7353.
33. Petersson, A., J. R. M. Almeida, T. Modig, K. Karhumaa, B. Hahn-Hagerdal, M. F. Gorwa-Grauslund, and G. Liden. 2006. A 5-hydroxymethyl furfural reducing enzyme encoded by Saccharomyces cerevisiae ADH6 gene conveys HMF tolerance. YEAST. 23:455-464.
34. Singh, N. P., and A. Khan. 1995. Acetaldehyde: Genotoxicity and cytotoxicity in human lymphocytes. Mutat. Res 337:9-17.
35. Sulzenbacher, G., K. Alvarez, R. Heuvel, C. Versluis, S. Spinelli, V. Campanacci, C. Valencia, C. Cambillau, H. Eklund, and M. Tegoni. 2004. Crystal structure of E. coli alcohol dehydrogenase YqhD: Evidence of a covalently modified NADP coenzyme. J. Mol. Biol. 342:489-502.
36. Talebnia, F., and M. J. Taherzadeh. 2006. In situ detoxification and continuous cultivation of dilute-acid hydrolysate to ethanol by encapsulated S-cerevisiae. J Biotechnol. 125:377-384.
37. Tokiwa, Y., and B. P. Calabia. 2008. Biological production of functional chemicals from renewable resources. Can. J. Chem. 86:548-555.
38. Urn, B. H., M. N. Karim, and L. L. Henk 2003. Effect of sulfuric and phosphoric acid pretreatments on enzymatic hydrolysis of corn stover. Appl Biochem Biotechnol. 105-108:115-125.
39. White, D. 2000. The Physiology and Biochemistry of Prokaryotes. 2^{nd} edition. Oxford University Press. New York, N.Y.
40. Almeida, J. R. M., M. Betilsson, M. F. Gorwa-Grauslund, S. Gorsich, and G. Liden. 2009. Metabolic effects of furaldehydes and impacts on biotechnological processes. Appl. Microbiol. Biotechnol. 82:625-638.
41. Amann, E., B. Ochs, and K. J. Abel. 1988. Tightly regulated tac promoter vectors for the expression of unfused and fused proteins in Escherichia coli. Gene 69:301-315.
42. Gama-Castro, S., J. J. Veronica, M. Peralta-Gil, A. Santos-Zavaleta, M. I. Peñaloza-Spindola, B. Contreras-Moreira, J. Segura-Salazar, L. Muñiz-Rascado, I. Martinez-Flores, H. Salgado, C. Bonavides-Martinez, C. Abreu-Goodger, C. Rodriguez-Penagos, J. Miranda-Rios, E. Morett, E. Merino, A. M. Huerta and J. Collado-Vides. 2008 RegulonDB (version 6.0): gene regulation model of Escherichia coli K-12 beyond transcription, active (experimental) annotated promoters and Textpresso navigation. Nucl. Acids Res., 36:D120-D124.
43. Gralla, J. D. 2005. Escherichia coli ribosomal RNA transcription: regulatory roles for ppGpp, NTPs, architectural proteins and a polymerase-binding protein. Molecular Microbiology. 55:973-977.
44. Gyaneshwar, P., O. Paliy, J. McAuliffe, D. L. Papham, M. I. Jordan, and S. Kustu. 2005. Sulfur and nitrogen limitation in Escherichia coli K-12: Specific homeostatic responses. J. Bacteriol. 187:1074-1090.
45. Hyduke, D. H., L. R. Jarboe, L. M. Tran, K. J. Y. Chou, and J. C. Liao. 2007. Integrated network analysis identifies nitric oxide response networks and dihydroxyacid dehydratase as a crucial target in Escherichia coli. PNAS. 104:8484-8489.
46. Liao, J. C., R. Boscolo, Y. L. Yang, L. M. Tran, C. Sabatti, and V. P. Roychowdhury. 2003. Network Component Analysis: Reconstruction of regulatory signals in biological systems. PNAS. 100:15522-15527.
47. Maier, T.H.2003. Semisynthetic production of L-alpha-amino acids by metabolic engineering of the cysteine-biosynthetic pathway. Nature Biotechnol. 4:422-427.
48. Miller, E. M., L. R. Jarboe, L. P. Yomano, S. W. York, K. T. Shanmugam and L. O. Ingram. 2009. Silencing of NADPH-dependent oxidoreductases (yqhD and dkgA) in furfural-resistant ethanologenic Escherichia coli. Appl. Environ. Microbiol. IN PRESS.
49. Nagasawa T., T. Ishi, H. Kumagai, and H. Yamada. 1985. D-cysteine desulfhydrase of Escherichia coli—purification and characterization. Eur. J. Biochem. 153:541-551.
50. Neidhardt, F. C. Escherichia coli and Salmonella Typhimurium. 1987. vol. 1. American Society for Microbiology. Washington, D.C.
51. Patten, C. L., M. G. Kirchhof, M. R. Schertzberg, R. A. Morton, and H. E. Schellhorn. 2004. Microarray analysis of RpoS-mediated gene expression in Escherichia coli K-12. Mol. Genet. Genomics. 272(5):580-591.
52. Sauer, U., F. Canonaco, S. Heri, A Perrenoud, E. Fisher. 2004. The soluble and membrane-bound transhydrogenases UdhA and PntAB have divergent functions in NADPH metabolism of Escherichia coli. J. Biol. Chem. 279:6613-6619.
53. Schomburg I., A. Chang, O. Hofmann, C. Ebeling, F. Ehrentreich, D. Schomburg. 2002. BRENDA: a resource for enzyme data and metabolic information. Trends Biochem. Sci. 27(1):54-6.
54. Siegel, L. M., P. S. Davis. 1974. Reduced Nicotinamide Adenine Dinucleotide Phosphate-Sulfite Reductase of Enterobacteria. J. Biol. Chem. 249:1587-1598.
55. Strom, A. R., and I. Kaasen. 1993. Trehalose metabolism in Escherichia coli: stress protection and stress regulation of gene expression. Mol. Microbiol. 8:205-210.
56. Tran, L. M., M. P. Brynildsen, K. C. Kao, J. K. Suen, and J. C. Liao. 2005. gNCA: a framework for determining transcription factor activity based on transcriptome: identifiability and numerical implementation. Metab Eng 7:128-41.
57. van der Ploeg, J. R., M. A. Weiss, E. Saller, H. Nahimoto, N. Saito, M. A. Kertesz, T. Leisinger. 1996. Identification of sulfate starvation-regulated genes in Escherichia coli: a gene cluster involved in the utilization of taurine as a sulfur source. J. Bacteriol. 178:5438-5446.
58. Vemuri, G. N., E. Altman, D. P. Sangurdekar, A. B. Khodursky, M. A. Eiteman. 2006. Overflow metabolism in Escherichia coli during steady-state growth: Transcriptional regulation and effect of the redox ratio. Appl. Environ. Microbiol. 72:3653-3661.
59. Weber, H., T. Polen, J. Heuveling, V. F. Wendish, and R. Hengge. 2005. Genome-wide analysis of the general stress response network in Escherichia coli: sigma S-dependent genes, promoters, and sigma factor selectivity. J. Bacteriol. 187:1591-1603.
60. Yomano, L. P., S. W. York, K. T. Shanmugam, and L. O. Ingram. 2009. Deletion of methylglyoxal synthase gene (mgsA) increased sugar co-metabolism in ethanol-producing Escherichia coli. Biotechnol. Lett. IN PRESS.
61. Zaldivar, J., and L. O. Ingram. 1999. Effect of organic acids on the growth and fermentation of ethanologenic Escherichia coli LY01. Biotechnol. Bioeng. 66: 203
62. Zaldivar, J., A. Martinez, and L. O. Ingram. 1999. Effect of selected aldehydes on the growth and fermentation of ethanologenic Escherichia coli. Biotechnol. Bioeng. 65: 24-33.
63. Zaldivar, J., A. Martinez, and L. O. Ingram. 2000. Effect of alcohol compounds found in hemicellulose hydrolysate on the growth and fermentation of ethanologenic Escherichia coli. Biotechnol. Bioeng. 68:524-530.
64. Zhang, T. F., and L. P. Hager. 1987. A single-step large scale purification of pyruvate oxidase. Arch. Biochem. Biophys. 257:485-48.
65. Almeida J R M, Roder A, Modig T, Laadan B, Liden G, Gorwa-Grauslund M F (2008) NADH- vs. NADPH-coupled reduction of 5-hydroxymethyl furfural (HMF) and its implications on product distribution in Saccharomyces cerevisiae. Appl Microbiol Biotechnol 78:939-945.
66. Cheng K K, Cai B Y, Zhang J A, Ling H Z, Zhou Y H, Ge J P, Xu J M (2008) Sugarcane bagasse hemicellulose hydrolysate for ethanol production by acid recovery process. Biochem Eng J 38:105-109.
67. Heer D, Heine D, Sauer U (2009) Resistance of Saccharomyces cerevisiae to high furfural concentration is based on NADPH-dependent reduction by at least two oxidoreductases. Appl Environ Microbiol doi:10.1128/AEM.01649-9.
68. Keseler I M, Bonavides-Martinez C, Collado-Vides J, Gama-Castro S, Gunsalus R P, Johnson D A, Krummenacker M, Nolan L M, Paley S, Paulsen I T, Peralta-Gil M, Santos-Zavaleta A, Shearer A G, Karp P D (2009) EcoCyc: a comprehensive view of Escherichia coli biology. Nucleic Acids Res 37:D464-70.
69. Liu Z L, Moon J (2009) A novel NADPH-dependent aldehyde reductase gene from Saccharomyces cerevisiae NRRL Y-12632 involved in the detoxification of aldehyde inhibitors derived from lignocellulosic biomass conversion. Gene 446:1-10.
70. Miller E N, Jarboe L R, Turner P C, Pharkya P, Yomano L P, York S W, Nunn D, Shanmugam K T, Ingram L O (2009a) Furfural inhibits growth by limiting sulfur assimilation in ethanologenic Escherichia coli strain LY180. Appl Environ Microbiol 75:6132-6141.
71. Purvis J E, Yomano L P, Ingram L O (2005) Enhanced trehalose production improves growth of Escherichia coli under osmotic stress. Appl Environ Microbiol 71:3761-3769.
72. Taherzadeh M J, Gustafsson L, Niklasson C, Liden G (2000) Physiological effects of 5-hydroxymethylfurfural on Saccharomyces cerevisiae. Appl Microbiol Biotechnol 53:701-708.
73. Um B H, Karim M N, Henk L L (2003) Effect of sulfuric and phosphoric acid pretreatments on enzymatic hydrolysis of corn stover. Appl Biochem Biotechnol 105:115-125.
74. Wyman C E, Dale B E, Elander R T, Holtzapple M, Ladisch M R, Lee Y Y (2005) Comparative sugar recovery data from laboratory scale application of leading pretreatment technologies to corn stover. Bioresour Technol, 96:2026-2032.

### INCORPORATION BY REFERENCE

All publications, patent applications and patents identified herein are expressly incorporated herein by reference in their entirety.

### EQUIVALENTS

Those skilled in the art will recognize, or be able to ascertain using no more than routine experimentation, many equivalents to the specific embodiments of the invention described herein. Such equivalents are intended to be encompassed by this invention.

1. An isolated or recombinant ethanologenic bacterium having increased expression of at least one of transhydrogenase genes pntA and pntB as compared to a reference bacterium, and having increased furfural tolerance as compared to a reference bacterium.

2. (canceled)

3. (canceled)

4. (canceled)

5. The bacterium of claim 3, wherein the reference bacterium is a wild-type bacterium.

6. The bacterium of claim 3, wherein said bacterium is ethanologenic.

7. The bacterium of claim 3, wherein said bacterium exhibits increased ethanol production as compared to a reference bacterium.

8. The bacterium of claim 3, wherein said bacterium exhibits increased ethanol production in the presence of furfural or 5-HMF as compared to a reference bacterium.

9. The bacterium of claim 3, where said bacterium has increased growth as compared to a reference bacterium.

10. The bacterium of claim 3, wherein said bacterium has increased growth in the presence of furfural or 5-HMF as compared to a reference bacterium.

11. The bacterium of claim 3, wherein said bacterium has increased growth in the presence of 5-HMF at concentrations between about 0.025% furfural to about 0.15% furfural.

12. The bacterium of claim 3, wherein said bacterium has increased growth in the presence of furfural at concentrations between about 0.025% furfural to about 0.15% furfural.

13. The bacterium of claim 3, wherein said bacterium has increased growth and increased ethanol production as compared to a reference bacterium.

14. The bacterium of claim 3, wherein said bacterium has increased growth in the presence of a hydrolysate as compared to a reference bacterium.

15. The bacterium of claim 14, wherein the hydrolysate is derived from a product comprising a biomass, a hemicellulosic biomass, a lignocellulosic biomass or a cellulosic biomass.

16. The bacterium of claim 3, wherein said expression is increased by modifying or adding a promoter or regulatory protein that regulates the expression of the pntA and pntB genes

17. The bacterium of claim 3, wherein said bacterium is capable of producing ethanol as a primary fermentation product under anaerobic or microaerobic conditions.

18. The bacterium of claim 3, wherein the bacterium is selected from the group consisting of Gram negative bacteria and Gram positive bacteria.

19. The bacterium of claim 18, wherein the Gram-negative bacterium is selected from the group consisting of Escherichia, Acinetobacter, Zymomonas, Gluconobacter, Geobacter, Shewanella, Salmonella, Enterobacter and Klebsiella.

20. The bacterium of claim 18, wherein the Gram-positive bacterium is selected from the group consisting of Bacillus, Clostridium, Corynebacterium, Lactobacillus, Lactococcus, Oenococcus, Streptococcus and Eubacterium.

21. The bacterium of claim 3, wherein the bacterium is Escherichia coli.

22. The bacterium of claim 3, wherein the bacterium is Klebsiella oxytoca.

23. An isolated or recombinant bacterium, wherein the activity of PntA and PntB proteins is increased as compared to a reference bacterium.

24. (canceled)

25. (canceled)

26. An isolated or recombinant bacterium, wherein expression of the pntA and pntB genes is increased as compared to a reference bacterium, and wherein the bacterium has increased furfural tolerance as compared to the reference bacterium.

27. An isolated or recombinant bacterium wherein the expression of the pntA and pntB genes or the activity of the PntA and PntB polypeptides is increased as compared to a reference bacterium, wherein furfural of 5-HMF tolerance is increased as compared to the reference bacterium, wherein said bacterium is capable of producing ethanol, and wherein the bacterium is prepared by a process comprising the steps of:
(a) growing a candidate strain of the bacterium in the presence of furfural or 5-HMF; and
(b) selecting bacterium that produces ethanol in the presence of furfural or 5-HMF.

28. A method for producing ethanol from a biomass, a hemicellulosic biomass, a lignocellulosic biomass, a cellulosic biomass or an oligosaccharide source comprising contacting the biomass, hemicellulosic biomass, lignocellulosic biomass, cellulosic biomass or oligosaccharide with the isolated or recombinant bacterium of claim 1 thereby producing ethanol from a biomass, hemicellulosic biomass, lignocellulosic biomass, cellulosic biomass or an oligosaccharide source.

29. A method for producing ethanol from a biomass, a hemicellulosic biomass, a lignocellulosic biomass, a cellulosic biomass or an oligosaccharide source in the presence of furfural comprising contacting the biomass, hemicellulosic biomass, lignocellulosic biomass, cellulosic biomass or oligosaccharide with the isolated or recombinant bacterium of claim 1, thereby producing ethanol from a biomass, hemicellulosic biomass, lignocellulosic biomass, cellulosic biomass or an oligosaccharide source.

30. Ethanol produced by the method of claim 28.

31. Ethanol produced by the method of claim 29.

32. (canceled)

33. The ethanologenic bacteria of claim 1, wherein the bacteria is the furfural-resistant mutant EMFR9.
